(19) United States
(12) Patent Application Publication
(10) Pub. No.: US 2022/0101971 A1
(43) Pub. Date: Mar. 31, 2022

(54) PREDICTIVE DATA ANALYSIS TECHNIQUES FOR GENERATING OPTIMIZED DRUG DELIVERY ARRANGEMENTS

(71) Applicant: Optum, Inc., Minnetonka, MN (US)

(72) Inventors: Jon Kevin Muse, Thompsons Station, TN (US); Gregory J. Boss, Saginaw, MI (US); Kate Hurley, Co. Dublin (IE); Rama S. Ravindranathan, Edison, NJ (US)

(21) Appl. No.: 17/034,049

(22) Filed: Sep. 28, 2020

(51) Int. Cl. G16H 20/10; G16H 10/60; G16H 50/70; G16H 70/40; G16H 50/30

(52) U.S. Cl. CPC G16H 20/10; G16H 10/60; G16H 50/30; G16H 70/40; G16H 50/70

(57) ABSTRACT

There is a need for more accurate and more efficient optimized delivery prediction operations. This need can be addressed by, for example, techniques for performing a plurality of optimized drug need prediction iterations in order to generate corresponding optimized delivery predictions. In one example, a method includes: performing a plurality of optimized drug need prediction iterations, wherein: each optimized drug need prediction iteration of the plurality of optimized drug need predictions is configured to generate an optimized delivery prediction for the optimized drug need prediction iteration; and performing the optimized drug delivery based at least in part on each optimized delivery prediction for an optimized drug need prediction iteration of the plurality of optimized drug need predictions.

### BACKGROUND

Various embodiments of the present invention address technical challenges related to generating optimized delivery predictions and performing optimized drug deliveries and disclose innovative techniques for improving efficiency and/or reliability of optimized delivery prediction systems.

### BRIEF SUMMARY

In general, embodiments of the present invention provide methods, apparatuses, systems, computing devices, computing entities, and/or the like for performing optimized delivery prediction operations. Various embodiments of the present invention disclose techniques for generating optimized delivery predictions that lead to determining recommended parameters and performing optimized drug deliveries.

In accordance with one aspect, a method for performing optimized drug delivery for a target drug profile with respect to a target user profile is provided. In one embodiment, the method comprises performing, by one or more computer processors, a plurality of optimized drug need prediction iterations, wherein: each optimized drug need prediction iteration of the plurality of optimized drug need predictions is configured to generate an optimized delivery prediction for the optimized drug need prediction iteration, generating the optimized delivery prediction for an initial optimized drug need prediction iteration of the plurality of optimized drug need predictions comprises: (i) determining an initial delivery dosage prediction for the initial optimized drug need prediction iteration based at least in part on a current medical history profile of the target user profile at a prediction time of the initial optimized drug need prediction iteration and of the target drug profile in relation to the target user profile, and (ii) generating the optimized delivery prediction for the initial optimized drug need prediction iteration based at least in part on the initial delivery dosage prediction, and generating each optimized delivery prediction for a post-initial optimized drug need prediction iteration of the plurality of optimized drug need predictions comprises: (i) determining a drug need prediction for the prediction time of the post-initial optimized drug need prediction iteration based at least in part on the drug necessity profile and current usage monitoring data for the target user profile in relation to the target drug profile during a target monitoring period for the post-initial optimized drug need prediction, (ii) in response to determining that the drug need prediction describes an affirmative drug need prediction, generating the optimized delivery prediction based at least in part on a post-initial delivery dosage prediction for the post-initial optimized drug need prediction iteration and a post-initial delivery timing prediction for the post-initial optimized drug need prediction iteration, and (iii) in response to determining that the drug need prediction describes a negative drug need prediction, generating the optimized delivery prediction based at least in part on the negative drug need prediction; and performing, by the one or more computer processors, the optimized drug delivery based at least in part on each optimized delivery prediction for an optimized drug need prediction iteration of the plurality of optimized drug need predictions.

In accordance with another aspect, an apparatus for performing optimized drug delivery for a target drug profile with respect to a target user profile is provided, the apparatus comprising at least one processor and at least one memory including program code, the at least one memory and the program code configured to, with the processor, cause the apparatus to at least: perform a plurality of optimized drug need prediction iterations, wherein: each optimized drug need prediction iteration of the plurality of optimized drug need predictions is configured to generate an optimized delivery prediction for the optimized drug need prediction iteration, generating the optimized delivery prediction for an initial optimized drug need prediction iteration of the plurality of optimized drug need predictions comprises: (i) determining an initial delivery dosage prediction for the initial optimized drug need prediction iteration based at least in part on a current medical history profile of the target user profile at a prediction time of the initial optimized drug need prediction iteration and of the target drug profile in relation to the target user profile, and (ii) generating the optimized delivery prediction for the initial optimized drug need prediction iteration based at least in part on the initial delivery dosage prediction, and generating each optimized delivery prediction for a post-initial optimized drug need prediction iteration of the plurality of optimized drug need predictions comprises: (i) determining a drug need prediction for the prediction time of the post-initial optimized drug need prediction iteration based at least in part on the drug necessity profile and current usage monitoring data for the target user profile in relation to the target drug profile during a target monitoring period for the post-initial optimized drug need prediction, (ii) in response to determining that the drug need prediction describes an affirmative drug need prediction, generating the optimized delivery prediction based at least in part on a post-initial delivery dosage prediction for the post-initial optimized drug need prediction iteration and a post-initial delivery timing prediction for the post-initial optimized drug need prediction iteration, and (iii) in response to determining that the drug need prediction describes a negative drug need prediction, generating the optimized delivery prediction based at least in part on the negative drug need prediction; and perform the optimized drug delivery based at least in part on each optimized delivery prediction for an optimized drug need prediction iteration of the plurality of optimized drug need predictions.

In accordance with yet another aspect, a computer program product for performing optimized drug delivery for a target drug profile with respect to a target user profile, the computer program product comprising at least one non-transitory computer-readable storage medium having computer-readable program code portions stored therein, the computer-readable program code portions configured to: perform a plurality of optimized drug need prediction iterations, wherein: each optimized drug need prediction iteration of the plurality of optimized drug need predictions is configured to generate an optimized delivery prediction for the optimized drug need prediction iteration, generating the optimized delivery prediction for an initial optimized drug need prediction iteration of the plurality of optimized drug need predictions comprises: (i) determining an initial delivery dosage prediction for the initial optimized drug need prediction iteration based at least in part on a current medical history profile of the target user profile at a prediction time of the initial optimized drug need prediction iteration and of the target drug profile in relation to the target user profile, and (ii) generating the optimized delivery prediction for the initial optimized drug need prediction iteration based at least in part on the initial delivery dosage prediction, and generating each optimized delivery prediction for a post-initial optimized drug need prediction iteration of the plurality of optimized drug need predictions comprises: (i) determining a drug need prediction for the prediction time of the post-initial optimized drug need prediction iteration based at least in part on the drug necessity profile and current usage monitoring data for the target user profile in relation to the target drug profile during a target monitoring period for the post-initial optimized drug need prediction, (ii) in response to determining that the drug need prediction describes an affirmative drug need prediction, generating the optimized delivery prediction based at least in part on a post-initial delivery dosage prediction for the post-initial optimized drug need prediction iteration and a post-initial delivery timing prediction for the post-initial optimized drug need prediction iteration, and (iii) in response to determining that the drug need prediction describes a negative drug need prediction, generating the optimized delivery prediction based at least in part on the negative drug need prediction; and perform the optimized drug delivery based at least in part on each optimized delivery prediction for an optimized drug need prediction iteration of the plurality of optimized drug need predictions.

### BRIEF DESCRIPTION OF THE DRAWINGS

Having thus described the invention in general terms, reference will now be made to the accompanying drawings, which are not necessarily drawn to scale, and wherein:

FIG. 1 provides an exemplary overview of a system architecture that can be used to practice embodiments of the present invention.

FIG. 2 provides an example optimized delivery prediction computing entity in accordance with some embodiments discussed herein.

FIG. 3 provides an example client computing entity in accordance with some embodiments discussed herein.

FIG. 4 provides an exemplary schematic of a system architecture for performing optimized delivery prediction operations and generating optimized delivery predictions in accordance with some embodiments discussed herein.

FIG. 5 provides a flowchart diagram illustrating an example process for generating an optimized delivery prediction for an initial optimized drug need prediction iteration in accordance with some embodiments discussed herein.

FIG. 6 provides a flowchart diagram illustrating an example process for generating an optimized delivery prediction for a post-initial optimized drug need prediction iteration in accordance with some embodiments discussed herein.

FIG. 7 provides a flowchart diagram illustrating an example process for determining a post-initial delivery dosage prediction in accordance with some embodiments discussed herein.

FIG. 8 provides a flowchart diagram illustrating an example process for generating a current medical history profile in accordance with some embodiments discussed herein.

FIGS. 9A-B provide operational examples of generating user interface data in accordance with some embodiments discussed herein.

FIGS. 10A-B provide flowchart diagrams illustrating an example user registration process in accordance with some embodiments discussed herein.

FIG. 11 provides a flowchart diagram illustrating an example optimized drug delivery process in accordance with some embodiments discussed herein.

### DETAILED DESCRIPTION

Various embodiments of the present invention are described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. The term “or” is used herein in both the alternative and conjunctive sense, unless otherwise indicated. The terms “illustrative” and “exemplary” are used to be examples with no indication of quality level. Like numbers refer to like elements throughout. Moreover, while certain embodiments of the present invention are described with reference to predictive data analysis, one of ordinary skill in the art will recognize that the disclosed concepts can be used to perform other types of data analysis.

### I. OVERVIEW

Various embodiments of the present invention disclose techniques for generating optimized delivery predictions and performing optimized drug deliveries that dynamically adjusts to changes in various conditions and parameters and improves the accuracy and reliability of generated predictions. Known delivery prediction systems are not configured to dynamically modify outputs/predictions in real-time based on the most up-to-date information. Additionally, much of the available data is often discarded and not used to enhance system reliability. There is a need for improved systems and methods configured to perform optimized drug need prediction iterations which utilize the output from each iteration in combination with up-to-date information about drug usage by the end-user. The inventors have confirmed, via experiments and theoretical calculations, that various embodiments of the disclosed techniques improve accuracy and reliability of optimized delivery prediction systems and predictive data analysis relative to various state-of-the-art solutions.

Various embodiments of the present invention perform optimized drug need prediction iterations in order to generate optimized delivery predictions that lead to generating recommended parameters of at least an initial delivery or a post-initial delivery. By using as input the output of previous iterations in combination with the most up-to-date information about drug usage by the end-user, the noted embodiments of the present invention disclose and enable a robust, dynamic system for providing optimized delivery predictions based on real-time information.

Using the methods described herein, the resulting optimized delivery prediction data outputs/objects contain more comprehensive information and lead to more accurate predictions which can be used to perform optimized drug deliveries and generate user interface data for an end user. Accordingly, by utilizing some or all of the innovative techniques disclosed herein for performing optimized delivery prediction operation, various embodiments of the present invention increase accuracy of drug-delivery-related parameter predictions. In doing so, various embodiments of the present invention make substantial technical contributions to the field of predictive data analysis and substantially improve state-of-the-art optimized delivery prediction systems.

### II. DEFINITIONS OF CERTAIN TERMS

The term “optimized drug need prediction iterations” may refer to a plurality of computer-implemented processes configured to generate one or more predictive outputs that lead to recommended parameters of at least an initial delivery, to a target user, of a recommended dosage of a drug at a recommended delivery time and one or more subsequent (i.e., post-initial) recommended dosages and corresponding recommended delivery times of the drug. Each iteration processes/includes current information associated with the target user, the drug and the previous iteration(s) in order to accurately determine optimal parameters for each subsequent recommended delivery time and corresponding recommended dosage for the target user (i.e., each optimized delivery prediction).

The term “optimized delivery prediction” may refer to a data object that describes a predictive output of one or more computer-implemented processes, wherein the predictive output describes one or more predictive inferences relating to an initial or a subsequent (i.e., post-initial) recommended delivery time and recommended dosage of a drug for a target user.

The term “initial delivery dosage prediction” may refer to a data object that describes a predictive output of one or more computer-implemented processes, wherein the predictive output describes a recommended dosage of a drug provided on an initial delivery based at least in part on a target user's current medical history profile and a drug necessity profile. The medical history profile may refer to a data object storing and/or providing access to historical medical information/data for the target user profile. In some embodiments, medical information/data can include age, gender, known health conditions, medical history, and/or the like. The medical information/data may further comprise, for example without limitation, one or more of a current prescribed medication count, a current per-condition prescribed medication count profile, a current co-morbidity count profile, a current serious condition profile, a current medication titration profile, a current medication adherence emergency profile, and a current high-risk genomic profile with respect to the target user. The drug necessity profile may refer to a data object storing and/or providing access to one or more drug characteristics (e.g., severe side-effects, high-risk of potential abuse and/or the like) and/or one or more user characteristics that render the drug necessary with respect to the target user profile (e.g., user age, one or more serious health conditions, and/or the like).

The term “post-initial delivery dosage prediction” may refer to a data object that describes a predictive output of one or more computer-implemented processes, wherein the predictive output describes a recommended dosage of a drug provided on a subsequent delivery based at least in part on a drug necessity profile and current usage monitoring data (e.g., target user drug usage data during a time period after the initial delivery, as well as a timing of the subsequent delivery). The current usage monitoring data may comprise information/data describing how often the target user profile takes the drug as prescribed (i.e., in accordance with a corresponding medication intake protocol/regimen). The drug necessity profile may comprise one or more drug characteristics (e.g., severe side-effects, high-risk of potential abuse and/or the like) and/or one or more user characteristics that render the drug necessary with respect to the target user profile (e.g., user age, one or more serious health conditions, and/or the like). The post-initial delivery dosage prediction may comprise a drug need prediction. The drug need prediction may refer to a data object that describes a predictive output, wherein the predictive output describes an inferred conclusion as to whether or not a target user needs a drug. The drug need prediction may be a negative drug need prediction or an affirmative drug need prediction. A negative drug need prediction may refer to a predictive output describing an inferred conclusion that a target user does not currently need and/or will no longer need a drug. A positive drug need prediction may refer to a predictive output describing an inferred conclusion that a target user currently needs a drug and/or will need a drug.

The term “medical history profile” may refer to a data object storing and/or providing access to historical medical information/data for a corresponding target user profile. In some embodiments, medical information/data can include age, gender, known health conditions, medical history, and/or the like. The medical information/data may further comprise, for example without limitation, one or more of a current prescribed medication count, a current per-condition prescribed medication count profile, a current co-morbidity count profile, a current serious condition profile, a current medication titration profile, a current medication adherence emergency profile, and a current high-risk genomic profile.

The term “prediction time” may refer to a data object that describes a time associated with performing an optimized drug need prediction iteration. For example, the prediction time of an initial delivery dosage predication may be an initial delivery time for a drug. The prediction time of a post-initial delivery dosage prediction may be a subsequent delivery time for the drug.

The term “drug need prediction” may refer to a data object that describes a predictive output, wherein the predictive output describes an inferred conclusion as to whether or not a target user needs a drug at a particular prediction time. The drug need prediction may be a negative drug need prediction or an affirmative drug need prediction. A negative drug need prediction may refer to a predictive output describing an inferred conclusion that a target user does not currently need and/or will no longer need a drug. A positive drug need prediction may refer to a predictive output describing an inferred conclusion that a target user currently needs a drug and/or will need a drug.

The term “current usage monitoring data” may refer to a data object storing and/or providing access to medication information/data describing medication usage by a target user over a period of time, where the period of time includes the time interval between a delivery time of an initial drug delivery and a delivery time of a subsequent drug delivery that is associated with the current user monitoring data. The current usage monitoring data may be or comprise drug usage data with respect to the target user profile for a period of time after an initial delivery (e.g., a target monitoring period). The current usage monitoring data may comprise information/data describing how often the target user profile takes the drug as prescribed and in accordance with a corresponding medication intake protocol/regimen.

The term “target monitoring period” may refer to a data object that describes a period of time corresponding with current usage monitoring data. The target monitoring period may be or include a time interval between a delivery time of an initial drug delivery and a delivery time of a subsequent drug delivery that is associated with the current usage monitoring data.

The term “affirmative drug need prediction” may refer to a data object that describes a predictive output of one or more computer-implemented processes, wherein the predictive output describes an inferred conclusion that a target user currently needs a drug and/or will need a drug.

The term “post-initial delivery timing prediction” may refer to a data object that describes a recommended delivery time for a subsequent (i.e., non-initial) drug delivery.

The term “negative drug need prediction” may refer to a data object that describes a predictive output, wherein the predictive output describes an inferred conclusion that a target user does not currently need and/or will no longer need a drug.

The term “current user condition severity score” may refer to a data object that describes a predictive output of one or more computer-implemented processes, wherein the predictive output describes a condition of a target user in relation to one or more current serious health conditions. The current user condition severity score may be determined based at least in part on the current medical history profile of the target user. The medical history profile may refer to a data object storing and/or providing access to historical medical information/data for the target user profile. In some embodiments, medical information/data can include age, gender, known health conditions, medical history, and/or the like. The medical information/data may further comprise, for example without limitation, one or more of a current prescribed medication count, a current per-condition prescribed medication count profile, a current co-morbidity count profile, a current serious condition profile, a current medication titration profile, a current medication adherence emergency profile, and a current high-risk genomic profile. In some embodiments, the current serious condition profile may comprise the current user condition severity score.

The term “current user dependence score” may refer to a data object that describes a predictive output of one or more computer-implemented processes, wherein the predictive output describes an inferred determination about a target user's dependence on a drug at a delivery time. The current user dependence score may be determined based at least in part on the current medical history profile of the target user. The medical history profile may refer to a data object storing and/or providing access to historical medical information/data for the target user profile. In some embodiments, medical information/data can include age, gender, known health conditions, medical history, and/or the like. The medical information/data may further comprise, for example without limitation, one or more of a current prescribed medication count, a current per-condition prescribed medication count profile, a current co-morbidity count profile, a current serious condition profile, a current medication titration profile, a current medication adherence emergency profile, and a current high-risk genomic profile. In some embodiments, the current user dependence score may be based at least in part on a current user condition severity score and a drug necessity profile. The current user condition severity score may refer to a predictive output that describes one or more serious health conditions of a target user at a delivery time. The drug necessity profile may refer to a data object storing and/or providing access to information/data with respect to one or more drug characteristics (e.g., severe side-effects, high-risk of potential abuse and/or the like) and/or one or more user characteristics that render the drug necessary with respect to the target user profile (e.g., user age, one or more serious health conditions, and/or the like).

The term “compliance score” may refer to a data object that describes a predictive output of one or more computer-implemented processes, wherein the predictive output describes a target user's adherence to a recommended drug/medication intake protocol/regimen for a drug over a period of time. The compliance score may be based at least in part on current usage monitoring data for the target user over the period of time. The term current usage monitoring data may refer to a data object storing and/or providing access to information/data with respect to medication usage by a target user over the period of time. For example, the current usage monitoring data may include one or more non-compliance events corresponding with the target user not taking a prescribed medication in accordance with a recommended medication intake protocol/regimen (e.g., taking a medication later than prescribed or missing a recommended dosage).

The term “sufficient non-compliance condition” may refer to a data object that describes a condition that satisfies one or more parameters with respect to a target user's compliance score, where the satisfaction of the condition describes that the target user has not adhered to a recommended drug/medication intake protocol/regimen. The compliance score may refer to a data object that describes a predictive output of one or more computer-implemented processes, wherein the predictive output describes the target user's adherence to a recommended drug/medication intake protocol/regimen for a drug over a period of time (e.g., subsequent to a delivery). The sufficient non-compliance condition may correspond with length of time, a number of missed dosages and/or the like. Different drugs may have different parameters in relation to the sufficient non-compliance condition. For example, a sufficient non-compliance condition for a drug to treat a serious health condition may be a single dosage of the drug, whereas a sufficient non-compliance condition for a drug to treat a less serious health condition may be several dosages and/or a longer length of time.

The term “sufficient lack of necessity condition” may refer to a data object that describes a condition of a target user corresponding with an inference that the target user does not currently and/or will no longer need a drug. The sufficient lack of necessity condition may be based at least in part on a drug necessity score and/or drug necessity profile for the target user. The drug necessity score may refer to a data object that describes whether a drug is considered necessary based at least in part on one or more drug characteristics (e.g., severe side-effects, high-risk of potential abuse and/or the like). The drug necessity profile may refer to a data object storing and/or providing access to the drug necessity score and/or one or more user characteristics that render the drug necessary with respect to the target user (e.g., user age, one or more serious health conditions, and/or the like). A negative drug need prediction may be determined based at least in part on a determination that a drug necessity score describes a sufficient lack of necessity condition.

The term “non-compliance events” may refer to a data object that describes one or more historical events or actions taken by a target user with respect to a recommended drug/medication protocol/regimen, where the non-compliance events describe lack of adherence to a recommended medication intake protocol/regimen for a corresponding drug/medication. For example, a non-compliance event may correspond with whether or not the target user took a prescribed medication in accordance with a recommended medication intake protocol/regimen or missed a recommended dosage of the drug. A compliance score may be determined based at least in part on historical occurrences of non-compliance events and/or a frequency with respect to non-compliance events. The compliance score may refer to a data object that describes a predictive output of one or more computer-implemented processes, wherein the predictive output describes a target user's adherence to a recommended medication intake protocol/regimen for a drug over a period of time after a particular delivery.

The term “estimated drug availability period” may refer to a data object that describes a predictive output of one or more computer-implemented processes, wherein the predictive output describes a prospective time period associated with remaining drug availability for a patient at a particular time. The estimated drug availability period may be based at least in part on a count of one or more non-compliance events. A compliance event may refer to a data object that describes one or more historical events or actions taken by a target user with respect to a recommended medication intake protocol/regimen. A non-compliance event may correspond with whether or not the target user took a prescribed medication in accordance with the recommended medication intake protocol/regimen or missed a recommended dosage. For example, if a target user misses one or more counts/dosages of a prescribed medication, the estimated drug availability period for the drug may be lengthened to correspond with the one or more count(s) that were not taken.

The term “optimal drug surplus period” may refer to a data object that describes a predictive output of one or more computer-implemented processes, wherein the predictive output describes a recommended period of recommended drug. The optimal drug surplus period may be based at least in part on the drug necessity score. The drug necessity score may refer to a data object that describes a predictive output that describes whether a drug is considered necessary based at least in part on one or more drug characteristics (e.g., severe side-effects, high-risk of potential abuse and/or the like). The estimated drug availability period may refer to a data object describing a predictive output of one or more computer-implemented processes, wherein the predictive output describes a prospective time period corresponding with an assumed number of remaining dosages available to a target user out of a total number of dosages from an initial or subsequent delivery.

The term “user availability profile” may refer to a data object storing and/or providing access to information/data relating to the prospective location of a target user across a time period. The prospective location information/data may include data from the target user's schedule (e.g., prospective travel plans). For example, the prospective location information/data may indicate that a target user intends to travel during a prospective time period. Accordingly, in may be inferred that the target user will be unavailable to receive (e.g., access, collect and/or the like) a delivery during the prospective time period. A post-initial delivery dosage prediction may be based at least in part on the target user profile availability profile such that additional dosages of a drug may be provided in a delivery to account for the prospective time period when the target user is expected to be unavailable to receive a subsequent delivery of the drug. The post-initial delivery dosage prediction may refer to a data object that describes a predictive output of one or more computer-implemented processes, wherein the predictive output describes a recommended dosage of a drug provided on a subsequent delivery based at least in part on a drug necessity profile and current usage monitoring data with respect to the target user during a subsequent time period after the initial delivery.

The term “current prescribed medication count” may refer to a data object for a count of a set of medications of a target user. The current prescribed medication count may comprise a total number of prescribed drugs and corresponding dosages for the target user. The current medical history profile of a target user may comprise the current prescribed medication count. The medical history profile may refer to a data object storing and/or providing access to historical medical information/data for the target user profile. In some embodiments, medical information/data can include age, gender, known health conditions, medical history, and/or the like. In some embodiments, a lower current prescribed medication count may be associated with a lower risk profile of the target user than a higher current prescribed medication count.

The term “current per-condition prescribed medication count” may refer to a data object describing a count of set of medications associated with a target user, where the set of medications are further associated with one or more health conditions of the target user. In some embodiments, more than one drug/medication may be associated with a particular health condition. In some cases, a health condition associated with a higher medication count may be indicative of a more serious health condition. The current medical history profile of a target user may comprise the current per-condition prescribed medication count. The medical history profile may refer to a data object storing and/or providing access to historical medical information/data for the target user profile. In some embodiments, medical information/data can include age, gender, known health conditions, medical history, and/or the like.

The term “current co-morbidity count” may refer to a data object storing and/or providing access to medication information/data for a target user, wherein the medication information/data describes a number of serious conditions of the target user. For example, the current co-morbidity count may indicate that a target user has one or more serious conditions associated with an increased risk of death (e.g., heart disease, cancer, stroke, Alzheimer's disease, diabetes, influenza, nephritis, intentional self harm/suicide and/or the like). The current medical history profile for a target user may comprise the current co-morbidity count. The medical history profile may refer to a data object storing and/or providing access to historical medical information/data for the target user profile. In some embodiments, medical information/data can include age, gender, known health conditions, medical history, and/or the like.

The term “current serious condition profile” may refer to a data object storing and/or providing access to medical information/data for a target user, wherein the medical information/data describes a likelihood that the target user has an increased risk of death due to a current co-morbidity count (e.g., a number of serious conditions of the target user). A target user with a current serious condition profile indicating certain serious conditions may have a corresponding high/increased risk with respect to prospective non-compliance events and/or any other type of medication interruptions than a target user having a current serious condition profile indicating few or no serious conditions. The current medical history profile for a target user may comprise the current serious condition profile. The medical history profile may refer to a data object storing and/or providing access to historical medical information/data for the target user profile. In some embodiments, medical information/data can include age, gender, known health conditions, medical history, and/or the like.

The term “current medication titration profile” may refer to a data object storing and/or providing access to medical information/data for a target user, wherein the medical information/data describes an administration frequency with respect to one or more drugs that a target user is taking. For example, a drug associated with a high administration frequency (e.g., five times a day) may be associated with a correspondingly high risk for the target user in comparison to a drug associated with a low administration frequency (e.g., once a week). The current medical history profile for a target user may comprise the current medication titration profile. The medical history profile may refer to a data object storing and/or providing access to historical medical information/data for the target user profile. In some embodiments, medical information/data can include age, gender, known health conditions, medical history, and/or the like.

The term “current medication adherence emergency profile” may refer to a data object storing and/or providing access to medical information/data for a target user, wherein the medical information/data describes a likelihood of a target user adhering to a medication intake protocol/regimen for a drug based at least in part on historical data describing compliance information. For example, the medical information/data may describe whether or not the target user frequently adheres to medication intake protocols/regimens for prescribed drugs. The current medical history profile for a target user may comprise the current medication adherence emergency profile. The medical history profile may refer to a data object storing and/or providing access to historical medical information/data for the target user profile. In some embodiments, medical information/data can include age, gender, known health conditions, medical history, and/or the like.

The term “current high-risk genomic profile” may refer to a data object storing and/or providing access to medical information/data for a target user, wherein the medical information/data describes a risk-profile of the target user based at least in part on genomic/genetic information of the target user. The current medical history profile for a target user may comprise the current high-risk genomic profile. The medical history profile may refer to a data object storing and/or providing access to historical medical information/data for the target user profile. In some embodiments, medical information/data can include age, gender, known health conditions, medical history, and/or the like.

### III. COMPUTER PROGRAM PRODUCTS, METHODS, AND COMPUTING ENTITIES

Embodiments of the present invention may be implemented in various ways, including as computer program products that comprise articles of manufacture. Such computer program products may include one or more software components including, for example, software objects, methods, data structures, or the like. A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform. Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, and/or a report writing language. In one or more example embodiments, a software component comprising instructions in one of the foregoing examples of programming languages may be executed directly by an operating system or other software component without having to be first transformed into another form. A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

A computer program product may include non-transitory computer-readable storage medium storing applications, programs, program modules, scripts, source code, program code, object code, byte code, compiled code, interpreted code, machine code, executable instructions, and/or the like (also referred to herein as executable instructions, instructions for execution, computer program products, program code, and/or similar terms used herein interchangeably). Such non-transitory computer-readable storage media include all computer-readable media (including volatile and non-volatile media).

In one embodiment, a non-volatile computer-readable storage medium may include a floppy disk, flexible disk, hard disk, solid-state storage (SSS) (e.g., a solid state drive (SSD), solid state card (SSC), solid state module (SSM), enterprise flash drive, magnetic tape, or any other non-transitory magnetic medium, and/or the like. A non-volatile computer-readable storage medium may also include a punch card, paper tape, optical mark sheet (or any other physical medium with patterns of holes or other optically recognizable indicia), compact disc read only memory (CD-ROM), compact disc-rewritable (CD-RW), digital versatile disc (DVD), Blu-ray disc (BD), any other non-transitory optical medium, and/or the like. Such a non-volatile computer-readable storage medium may also include read-only memory (ROM), programmable read-only memory (PROM), erasable programmable read-only memory (EPROM), electrically erasable programmable read-only memory (EEPROM), flash memory (e.g., Serial, NAND, NOR, and/or the like), multimedia memory cards (MMC), secure digital (SD) memory cards, SmartMedia cards, CompactFlash (CF) cards, Memory Sticks, and/or the like. Further, a non-volatile computer-readable storage medium may also include conductive-bridging random access memory (CBRAM), phase-change random access memory (PRAM), ferroelectric random-access memory (FeRAIVI), non-volatile random-access memory (NVRAM), magnetoresistive random-access memory (MRAM), resistive random-access memory (RRAM), Silicon-Oxide-Nitride-Oxide-Silicon memory (SONOS), floating junction gate random access memory (FJG RAM), Millipede memory, racetrack memory, and/or the like.

In one embodiment, a volatile computer-readable storage medium may include random access memory (RAM), dynamic random access memory (DRAM), static random access memory (SRAM), fast page mode dynamic random access memory (FPM DRAM), extended data-out dynamic random access memory (EDO DRAM), synchronous dynamic random access memory (SDRAM), double data rate synchronous dynamic random access memory (DDR SDRAM), double data rate type two synchronous dynamic random access memory (DDR2 SDRAM), double data rate type three synchronous dynamic random access memory (DDR3 SDRAM), Rambus dynamic random access memory (RDRAM), Twin Transistor RAM (TTRAM), Thyristor RAM (T-RAM), Zero-capacitor (Z-RAM), Rambus in-line memory module (RIMM), dual in-line memory module (DIMM), single in-line memory module (SIMM), video random access memory (VRAM), cache memory (including various levels), flash memory, register memory, and/or the like. It will be appreciated that where embodiments are described to use a computer-readable storage medium, other types of computer-readable storage media may be substituted for or used in addition to the computer-readable storage media described above.

As should be appreciated, various embodiments of the present invention may also be implemented as methods, apparatuses, systems, computing devices, computing entities, and/or the like. As such, embodiments of the present invention may take the form of an apparatus, system, computing device, computing entity, and/or the like executing instructions stored on a computer-readable storage medium to perform certain steps or operations. Thus, embodiments of the present invention may also take the form of an entirely hardware embodiment, an entirely computer program product embodiment, and/or an embodiment that comprises combination of computer program products and hardware performing certain steps or operations.

Embodiments of the present invention are described below with reference to block diagrams and flowchart illustrations. Thus, it should be understood that each block of the block diagrams and flowchart illustrations may be implemented in the form of a computer program product, an entirely hardware embodiment, a combination of hardware and computer program products, and/or apparatuses, systems, computing devices, computing entities, and/or the like carrying out instructions, operations, steps, and similar words used interchangeably (e.g., the executable instructions, instructions for execution, program code, and/or the like) on a computer-readable storage medium for execution. For example, retrieval, loading, and execution of code may be performed sequentially such that one instruction is retrieved, loaded, and executed at a time. In some exemplary embodiments, retrieval, loading, and/or execution may be performed in parallel such that multiple instructions are retrieved, loaded, and/or executed together. Thus, such embodiments can produce specifically-configured machines performing the steps or operations specified in the block diagrams and flowchart illustrations. Accordingly, the block diagrams and flowchart illustrations support various combinations of embodiments for performing the specified instructions, operations, or steps.

### IV. EXEMPLARY SYSTEM ARCHITECTURE

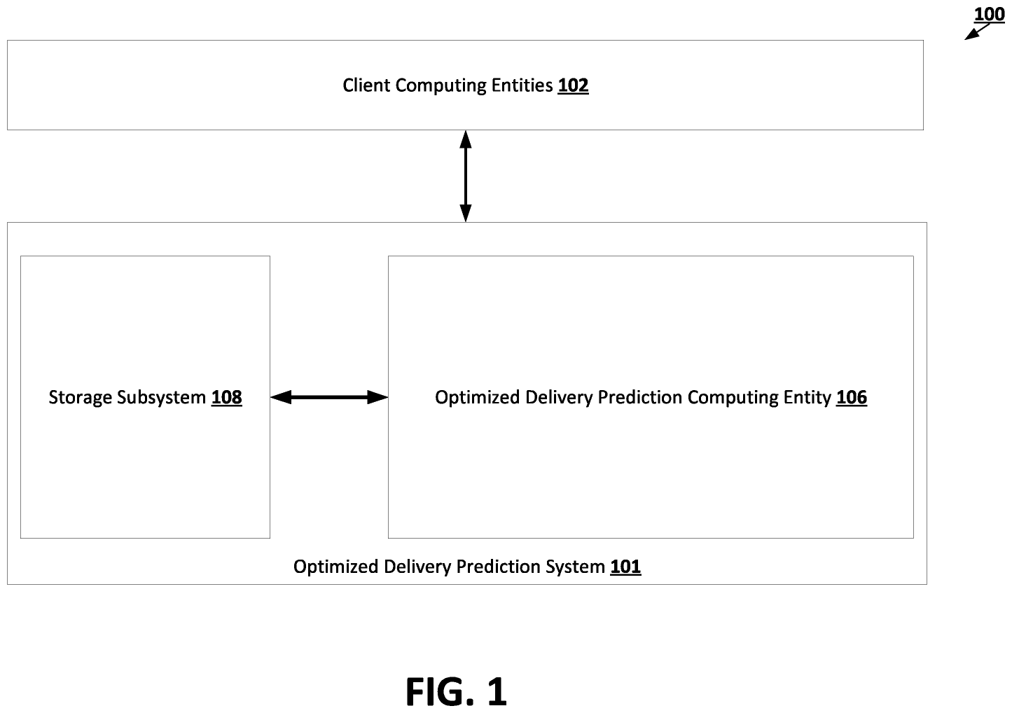

FIG. 1 is a schematic diagram of an example system architecture 100 for performing optimized delivery prediction operations. The architecture 100 includes a optimized delivery prediction system 101 configured to receive data from the client computing entities 102, process the data to generate predictive outputs (e.g., optimized delivery prediction data objects) and provide the outputs to the client computing entities 102 for generating user interface data and/or dynamically updating a user interface. In some embodiments, optimized delivery prediction system 101 may communicate with at least one of the client computing entities 102 using one or more communication networks. Examples of communication networks include any wired or wireless communication network including, for example, a wired or wireless local area network (LAN), personal area network (PAN), metropolitan area network (MAN), wide area network (WAN), or the like, as well as any hardware, software and/or firmware required to implement it (such as, e.g., network routers, and/or the like).

The optimized delivery prediction system 101 may include an optimized delivery prediction computing entity 106 and a storage subsystem 108. The optimized delivery prediction computing entity 106 may be configured to receive queries, requests and/or data from client computing entities 102, process the queries, requests and/or data to generate predictive outputs, and provide (e.g., transmit, send and/or the like) the predictive outputs to the client computing entities 102. The client computing entities 102 may be configured to transmit requests to the optimized delivery prediction computing entity 106 in response to events which satisfy certain parameters (e.g., monitored events). Responsive to receiving the predictive outputs, the client computing entities 102 may generate user interface data and may provide (e.g., transmit, send and/or the like) the target user profile interface data for presentation by user computing entities.

The storage subsystem 108 may be configured to store at least a portion of the data utilized by the optimized delivery prediction computing entity 106 to perform optimized delivery prediction operations and tasks. The storage subsystem 108 may be configured to store at least a portion of operational data and/or operational configuration data including operational instructions and parameters utilized by the optimized delivery prediction computing entity 106 to perform optimized delivery prediction operations in response to requests. The storage subsystem 108 may include one or more storage units, such as multiple distributed storage units that are connected through a computer network. Each storage unit in the storage subsystem 108 may store at least one of one or more data assets and/or one or more data about the computed properties of one or more data assets. Moreover, each storage unit in the storage subsystem 108 may include one or more non-volatile storage or memory media including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

### Exemplary Optimized Delivery Prediction Computing Entity

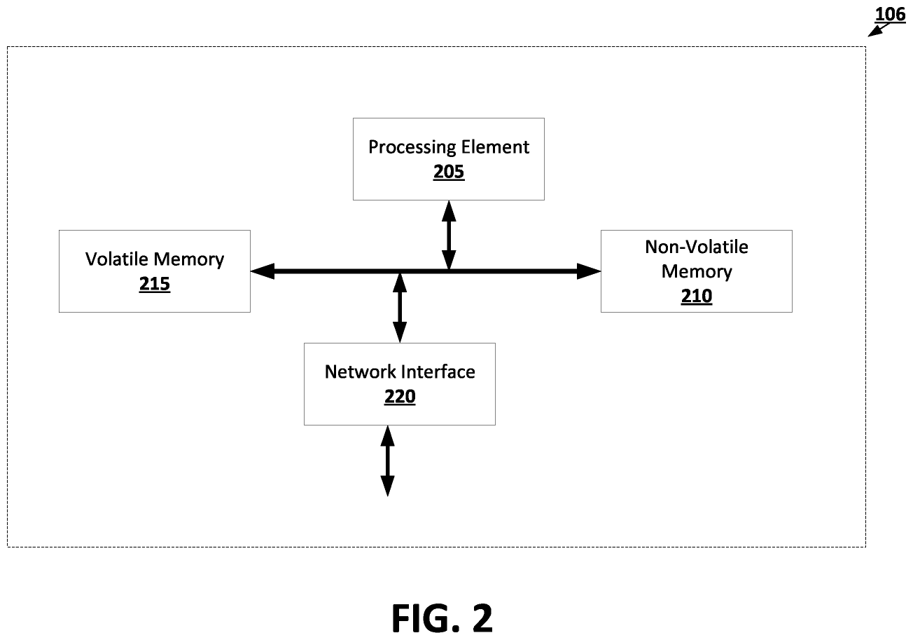

FIG. 2 provides a schematic of a optimized delivery prediction computing entity 106 according to one embodiment of the present invention. In general, the terms computing entity, computer, entity, device, system, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Such functions, operations, and/or processes may include, for example, transmitting, receiving, operating on, processing, displaying, storing, determining, creating/generating, monitoring, evaluating, comparing, and/or similar terms used herein interchangeably. In one embodiment, these functions, operations, and/or processes can be performed on data, content, information, and/or similar terms used herein interchangeably.

As indicated, in one embodiment, the optimized delivery prediction computing entity 106 may also include one or more network interfaces 220 for communicating with various computing entities, such as by communicating data, content, information, and/or similar terms used herein interchangeably that can be transmitted, received, operated on, processed, displayed, stored, and/or the like.

As shown in FIG. 2, in one embodiment, the optimized delivery prediction computing entity 106 may include or be in communication with one or more processing elements 205 (also referred to as processors, processing circuitry, and/or similar terms used herein interchangeably) that communicate with other elements within the optimized delivery prediction computing entity 106 via a bus, for example. As will be understood, the processing element 205 may be embodied in a number of different ways.

For example, the processing element 205 may be embodied as one or more complex programmable logic devices (CPLDs), microprocessors, multi-core processors, coprocessing entities, application-specific instruction-set processors (ASIPs), microcontrollers, and/or controllers. Further, the processing element 205 may be embodied as one or more other processing devices or circuitry. The term circuitry may refer to an entirely hardware embodiment or a combination of hardware and computer program products. Thus, the processing element 205 may be embodied as integrated circuits, application specific integrated circuits (ASICs), field programmable gate arrays (FPGAs), programmable logic arrays (PLAs), hardware accelerators, other circuitry, and/or the like.

As will therefore be understood, the processing element 205 may be configured for a particular use or configured to execute instructions stored in volatile or non-volatile media or otherwise accessible to the processing element 205. As such, whether configured by hardware or computer program products, or by a combination thereof, the processing element 205 may be capable of performing steps or operations according to embodiments of the present invention when configured accordingly.

In one embodiment, the optimized delivery prediction computing entity 106 may further include or be in communication with non-volatile media (also referred to as non-volatile storage, memory, memory storage, memory circuitry and/or similar terms used herein interchangeably). In one embodiment, the non-volatile storage or memory may include one or more non-volatile storage or memory media 210, including but not limited to hard disks, ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like.

As will be recognized, the non-volatile storage or memory media may store databases, database instances, database management systems, data, applications, programs, program modules, scripts, source code, object code, byte code, compiled code, interpreted code, machine code, executable instructions, and/or the like. The term database, database instance, database management system, and/or similar terms used herein interchangeably may refer to a collection of records or data that is stored in a computer-readable storage medium using one or more database models, such as a hierarchical database model, network model, relational model, entity—relationship model, object model, document model, semantic model, graph model, and/or the like.

In one embodiment, the optimized delivery prediction computing entity 106 may further include or be in communication with volatile media (also referred to as volatile storage, memory, memory storage, memory circuitry and/or similar terms used herein interchangeably). In one embodiment, the volatile storage or memory may also include one or more volatile storage or memory media 215, including but not limited to RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like.

As will be recognized, the volatile storage or memory media may be used to store at least portions of the databases, database instances, database management systems, data, applications, programs, program modules, scripts, source code, object code, byte code, compiled code, interpreted code, machine code, executable instructions, and/or the like being executed by, for example, the processing element 205. Thus, the databases, database instances, database management systems, data, applications, programs, program modules, scripts, source code, object code, byte code, compiled code, interpreted code, machine code, executable instructions, and/or the like may be used to control certain aspects of the operation of the optimized delivery prediction computing entity 106 with the assistance of the processing element 205 and operating system.

As indicated, in one embodiment, the optimized delivery prediction computing entity 106 may also include one or more network interfaces 220 for communicating with various computing entities, such as by communicating data, content, information, and/or similar terms used herein interchangeably that can be transmitted, received, operated on, processed, displayed, stored, and/or the like. Such communication may be executed using a wired data transmission protocol, such as fiber distributed data interface (FDDI), digital subscriber line (DSL), Ethernet, asynchronous transfer mode (ATM), frame relay, data over cable service interface specification (DOC SIS), or any other wired transmission protocol. Similarly, the optimized delivery prediction computing entity 106 may be configured to communicate via wireless client communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra-wideband (UWB), infrared (IR) protocols, near field communication (NFC) protocols, Wibree, Bluetooth protocols, wireless universal serial bus (USB) protocols, and/or any other wireless protocol.

Although not shown, the optimized delivery prediction computing entity 106 may include or be in communication with one or more input elements, such as a keyboard input, a mouse input, a touch screen/display input, motion input, movement input, audio input, pointing device input, joystick input, keypad input, and/or the like. The optimized delivery prediction computing entity 106 may also include or be in communication with one or more output elements (not shown), such as audio output, video output, screen/display output, motion output, movement output, and/or the like.

### Exemplary Client Computing Entity

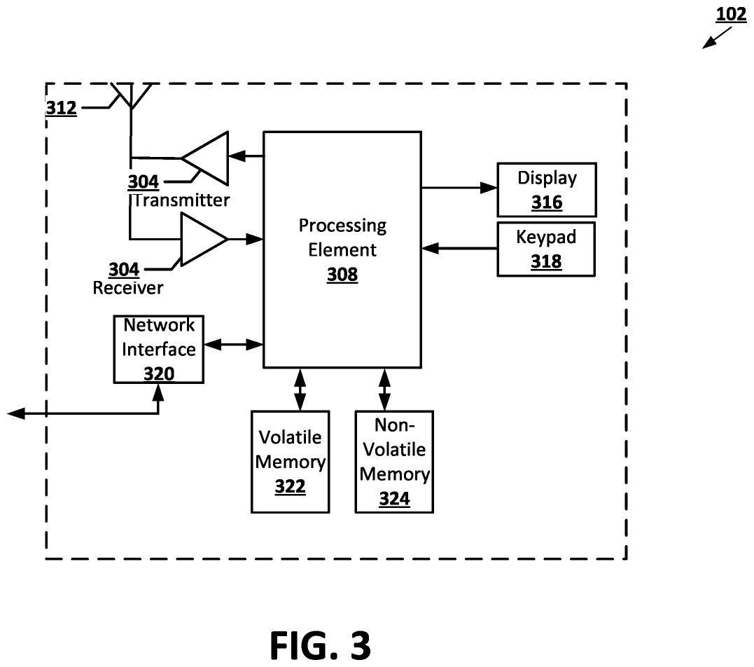

FIG. 3 provides an illustrative schematic representative of a client computing entity 102 that can be used in conjunction with embodiments of the present invention. In general, the terms device, system, computing entity, entity, and/or similar words used herein interchangeably may refer to, for example, one or more computers, computing entities, desktops, mobile phones, tablets, phablets, notebooks, laptops, distributed systems, kiosks, input terminals, servers or server networks, blades, gateways, switches, processing devices, processing entities, set-top boxes, relays, routers, network access points, base stations, the like, and/or any combination of devices or entities adapted to perform the functions, operations, and/or processes described herein. Client computing entities 102 can be operated by various parties. As shown in FIG. 3, the client computing entity 102 can include an antenna 312, a transmitter 304 (e.g., radio), a receiver 306 (e.g., radio), and a processing element 308 (e.g., CPLDs, microprocessors, multi-core processors, coprocessing entities, ASIPs, microcontrollers, and/or controllers) that provides signals to and receives signals from the transmitter 304 and receiver 306, correspondingly.

The signals provided to and received from the transmitter 304 and the receiver 306, correspondingly, may include signaling information/data in accordance with air interface standards of applicable wireless systems. In this regard, the client computing entity 102 may be capable of operating with one or more air interface standards, communication protocols, modulation types, and access types. More particularly, the client computing entity 102 may operate in accordance with any of a number of wireless communication standards and protocols, such as those described above with regard to the optimized delivery prediction computing entity 106. In a particular embodiment, the client computing entity 102 may operate in accordance with multiple wireless communication standards and protocols, such as UMTS, CDMA2000, 1×RTT, WCDMA, GSM, EDGE, TD-SCDMA, LTE, E-UTRAN, EVDO, HSPA, HSDPA, Wi-Fi, Wi-Fi Direct, WiMAX, UWB, IR, NFC, Bluetooth, USB, and/or the like. Similarly, the client computing entity 102 may operate in accordance with multiple wired communication standards and protocols, such as those described above with regard to the optimized delivery prediction computing entity 106 via a network interface 320.

Via these communication standards and protocols, the client computing entity 102 can communicate with various other entities using concepts such as Unstructured Supplementary Service Data (USSD), Short Message Service (SMS), Multimedia Messaging Service (MIMS), Dual-Tone Multi-Frequency Signaling (DTMF), and/or Subscriber Identity Module Dialer (SIM dialer). The client computing entity 102 can also download changes, add-ons, and updates, for instance, to its firmware, software (e.g., including executable instructions, applications, program modules), and operating system.

According to one embodiment, the client computing entity 102 may include location determining aspects, devices, modules, functionalities, and/or similar words used herein interchangeably. For example, the client computing entity 102 may include outdoor positioning aspects, such as a location module adapted to acquire, for example, latitude, longitude, altitude, geocode, course, direction, heading, speed, universal time (UTC), date, and/or various other information/data. In one embodiment, the location module can acquire data, sometimes known as ephemeris data, by identifying the number of satellites in view and the relative positions of those satellites (e.g., using global positioning systems (GPS)). The satellites may be a variety of different satellites, including Low Earth Orbit (LEO) satellite systems, Department of Defense (DOD) satellite systems, the European Union Galileo positioning systems, the Chinese Compass navigation systems, Indian Regional Navigational satellite systems, and/or the like. This data can be collected using a variety of coordinate systems, such as the Decimal Degrees (DD); Degrees, Minutes, Seconds (DMS); Universal Transverse Mercator (UTM); Universal Polar Stereographic (UPS) coordinate systems; and/or the like. Alternatively, the location information/data can be determined by triangulating the client computing entity's 102 position in connection with a variety of other systems, including cellular towers, Wi-Fi access points, and/or the like. Similarly, the client computing entity 102 may include indoor positioning aspects, such as a location module adapted to acquire, for example, latitude, longitude, altitude, geocode, course, direction, heading, speed, time, date, and/or various other information/data. Some of the indoor systems may use various position or location technologies including RFID tags, indoor beacons or transmitters, Wi-Fi access points, cellular towers, nearby computing devices (e.g., smartphones, laptops) and/or the like. For instance, such technologies may include the iBeacons, Gimbal proximity beacons, Bluetooth Low Energy (BLE) transmitters, NFC transmitters, and/or the like. These indoor positioning aspects can be used in a variety of settings to determine the location of someone or something to within inches or centimeters.

The client computing entity 102 may also comprise a user interface (that can include a display 316 coupled to a processing element 308) and/or a user input interface (coupled to a processing element 308). For example, the target user profile interface may be a user application, browser, user interface, and/or similar words used herein interchangeably executing on and/or accessible via the client computing entity 102 to interact with and/or cause display of information/data from the optimized delivery prediction computing entity 106, as described herein. The target user profile input interface can comprise any of a number of devices or interfaces allowing the client computing entity 102 to receive data, such as a keypad 318 (hard or soft), a touch display, voice/speech or motion interfaces, or other input device. In embodiments including a keypad 318, the keypad 318 can include (or cause display of) the conventional numeric (0-9) and related keys (#, *), and other keys used for operating the client computing entity 102 and may include a full set of alphabetic keys or set of keys that may be activated to provide a full set of alphanumeric keys. In addition to providing input, the target user profile input interface can be used, for example, to activate or deactivate certain functions, such as screen savers and/or sleep modes.

The client computing entity 102 can also include volatile storage or memory 322 and/or non-volatile storage or memory 324, which can be embedded and/or may be removable. For example, the non-volatile memory may be ROM, PROM, EPROM, EEPROM, flash memory, MMCs, SD memory cards, Memory Sticks, CBRAM, PRAM, FeRAM, NVRAM, MRAM, RRAM, SONOS, FJG RAM, Millipede memory, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, TTRAM, T-RAM, Z-RAM, RIMM, DIMM, SIMM, VRAM, cache memory, register memory, and/or the like. The volatile and non-volatile storage or memory can store databases, database instances, database management systems, data, applications, programs, program modules, scripts, source code, object code, byte code, compiled code, interpreted code, machine code, executable instructions, and/or the like to implement the functions of the client computing entity 102. As indicated, this may include a user application that is resident on the entity or accessible through a browser or other user interface for communicating with the optimized delivery prediction computing entity 106 and/or various other computing entities.

In another embodiment, the client computing entity 102 may include one or more components or functionality that are the same or similar to those of the optimized delivery prediction computing entity 106, as described in greater detail above. As will be recognized, these architectures and descriptions are provided for exemplary purposes only and are not limiting to the various embodiments.

In various embodiments, the client computing entity 102 may be embodied as an artificial intelligence (AI) computing entity, such as an Amazon Echo, Amazon Echo Dot, Amazon Show, Google Home, and/or the like. Accordingly, the client computing entity 102 may be configured to provide and/or receive information/data from a user via an input/output mechanism, such as a display, a camera, a speaker, a voice-activated input, and/or the like. In certain embodiments, an AI computing entity may comprise one or more predefined and executable program algorithms stored within an onboard memory storage module, and/or accessible over a network. In various embodiments, the AI computing entity may be configured to retrieve and/or execute one or more of the predefined program algorithms upon the occurrence of a predefined trigger event.

### V. EXEMPLARY SYSTEM OPERATIONS

Described herein are various techniques for performing optimized delivery prediction operations. Some of the described techniques utilize a particular configuration of units and/or iterations. The output of an optimized drug prediction operation may be supplied as an input for subsequent optimized drug prediction operations. However, a person of ordinary skill in the art will recognize that optimized delivery prediction operations discussed herein may be performed using different combinations than the particular combinations described herein.

By facilitating efficient optimized delivery prediction operations, various embodiments of the present invention improve accuracy of generated optimized delivery predictions. Performing optimized drug deliveries according to the methods disclosed facilitates dynamic updates in response to changing conditions and parameters. This in turn ensures that available monitoring data is utilized to improve system reliability and efficiency.

### Exemplary Optimized Delivery Prediction System

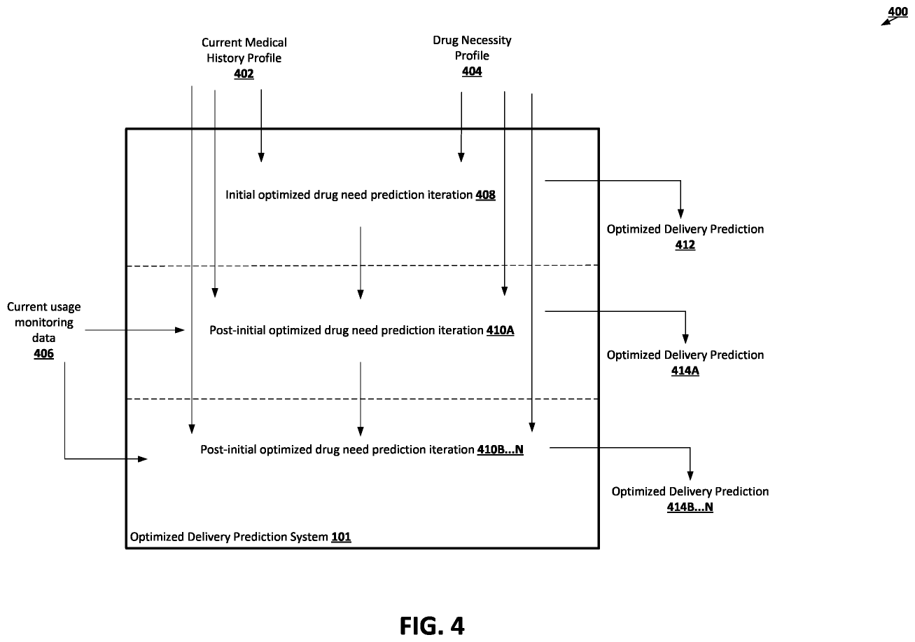

FIG. 4 provides an exemplary optimized delivery prediction system architecture 400. The optimized delivery prediction system 101 is configured to perform at least an initial optimized drug need prediction iteration 408, and a plurality of post-initial optimized drug need prediction iterations 410A, 410B . . . N in order to generate one or more predictive outputs. The term optimized drug need prediction iteration may refer to a plurality of computer-implemented processes configured to generate one or more predictive outputs that lead to recommended parameters of at least an initial delivery, to a target user, of a recommended dosage of a drug at a recommended delivery time and one or more subsequent (i.e., post-initial) recommended dosages and corresponding recommended delivery times of the drug. Each iteration processes/includes current information associated with the target user, the drug and the previous iteration(s) in order to accurately determine optimal parameters for each subsequent recommended delivery time and corresponding recommended dosage for the target user (i.e., each optimized delivery prediction). As shown, the optimized delivery prediction system 101 is configured to perform the initial optimized drug need prediction iteration 408 and the plurality of post-initial optimized drug need prediction iterations 410A, 410B . . . N in order to generate an initial optimized delivery prediction 412 and a plurality of optimized delivery predictions 414A, 414B . . . N. The term optimized delivery prediction may refer to a data object that describes a predictive output of one or more computer-implemented processes, wherein the predictive output describes one or more predictive inferences relating to an initial or a subsequent (i.e., post-initial) recommended delivery time and recommended dosage of a drug for a target user.

The storage subsystem 108 may provide, as input to the optimized delivery prediction computing entity 106, current usage monitoring data 406 for the target user profile with respect to the target drug profile, current medical history profile 402 for the target user profile, current drug necessity profile 404 for the target user profile with respect to the target drug profile, and/or the like. As shown, the optimized delivery prediction system 101 is configured to perform an initial optimized drug need prediction iteration 408 based least in part on the current medical history profile 402 and the current drug necessity profile 404.

The current medical history profile 402 may describe historical medical information/data for the target user profile. In some embodiments, medical information/data can describe at least one of age, gender, known health conditions, medical history, and/or the like. The drug necessity profile 404 may describe one or more drug characteristics (e.g., severe side-effects, high-risk of potential abuse and/or the like) and/or one or more user characteristics that render the drug necessary with respect to the target user profile (e.g., user age, one or more serious health conditions, and/or the like). For example, a drug that is not generally classified as a necessary drug may be necessary for a target user due to other considerations including age, comorbidities and/or the like.

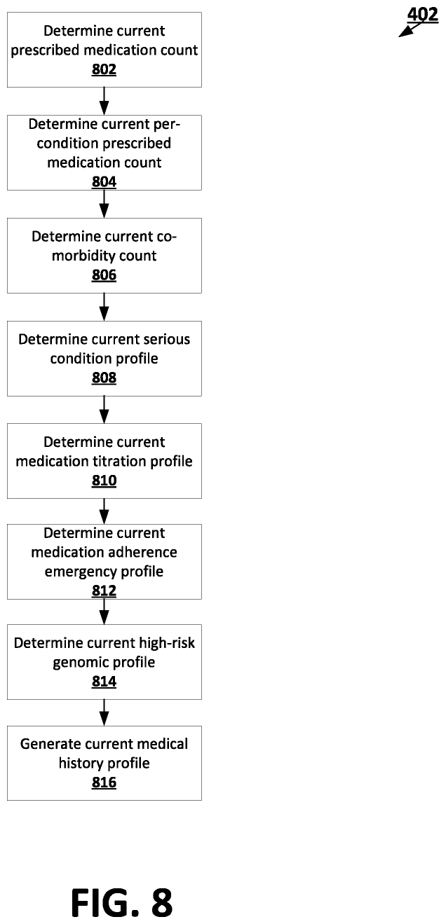

FIG. 8 provides a flowchart diagram illustrating an example process for generating a current medical history profile 402 in accordance with some embodiments discussed herein. It should be understood that while the description below provides an example process for generating a current medical history profile 402, the scope of the present disclosure is not limited to the description below and may comprise one or more additional and/or alternative elements and/or steps/operations.

Beginning at step/operation 802, the optimized delivery prediction system 101 determines a current prescribed medication count for the target user profile, e.g., a count of a set of medications for the target user. The current prescribed medication count may be a total number of prescribed drugs and corresponding dosages for the target user profile. A target user profile associated with a lower current prescribed medication count (i.e., lower number of prescribed drugs and dosages) may have a lower risk profile than a target user profile than a higher current prescribed medication count.

At step/operation 804, the optimized delivery prediction system 101 determines a current per-condition prescribed medication count for the target user profile. The current per-condition prescribed medication count may be a count of a set of medications reflecting a number of medications that are used to treat the same health condition. For example, a particular health condition associated with a high number of prescribed medications may indicate that the health condition is more serious in contrast with a health condition associated with few medications. Further, a health condition corresponding with a high number of prescribed medications may be indicative of a more serious health condition and/or more risk in relation to the target user's medication intake protocol/regimen (e.g., a higher likelihood that the prescriber has accounted for contraindications and potential side effects).

At step/operation 806, the optimized delivery prediction system 101 determines a current co-morbidity count for the target user profile. The current co-morbidity count may be the number of serious health conditions of the target user that are associated with an increased risk of death. Serious health conditions can also be leading causes of death (LCOD) for a target user based at least in part on characteristics such as age, gender, and/or the like. Example serious health conditions include heart disease, cancer, stroke, Alzheimer's disease, diabetes, influenza, nephritis, intentional self harm/suicide and/or the like.

At step/operation 808, the optimized delivery prediction system 101 determines a current serious condition profile for the target user profile. A current serious condition profile may comprise medical information/data describing a likelihood that the target user has an increased risk of death due to a current co-morbidity count (e.g., a number of serious health conditions that is a leading cause of death such as heart disease, cancer and/or the like). A current serious condition profile including certain serious conditions may indicate a corresponding high/increased risk of prospective non-compliance events and/or any other type of medication interruptions than a current serious condition profile indicating few or no serious health conditions. In other words, a target user with serious health conditions such as heart disease may be more adversely impacted by missed dosages of medication than a target user without serious health conditions.

At step/operation 810, the optimized delivery prediction system 101 determines a current medication titration profile for the target user profile. The current medication titration profile may comprise medical information/data describing an administration frequency with respect to one or more drugs that the target user is taking. For instance, a drug that is taken frequently (e.g., five times a day) may be indicative of a determination that the target user needs the drug and may further be indicative of a correspondingly high risk for the target user in comparison to a drug that is taken less frequently (e.g., once a week).

At step/operation 812, the optimized delivery prediction system 101 determines a current medication adherence emergency profile for the target user profile. The current medication adherence emergency profile may be a likelihood of a target user adhering to a medication intake protocol/regimen for a drug based at least in part on historical data describing compliance information. For example, the medical information/data may include historical data including incidents of emergency medical treatment related to prescription medication adherence such as overdosing, underdosing, medication adjustment failures and/or the like.

At step/operation 814, the optimized delivery prediction system 101 determines a current high-risk genomic profile for the target user profile. The current high-risk genomic profile may comprise a risk-profile of the target user based at least in part on genomic/genetic information of the target user. For example, the target user has certain genomes that indicate a high/elevated risk of contracting a given health condition such that improper medication intake protocols/regimens may be ineffective and/or accelerate progress of the health condition in the target user.

Then, at step/operation 816, the optimized delivery prediction system 101 determines the current medical history profile 402 based at least in part on the current prescribed medication count, the current per-condition prescribed medication count, the current co-morbidity count, the current serious condition profile, the current medication titration profile, the current medication adherence emergency profile, and the current high-risk genomic profile with respect to the target user. The current medical history profile may comprise an aggregated risk score in which each of the aforementioned determinations/factors (e.g., current prescribed medication count and the like) is associated with a respective score/value and/or weight in the aggregated risk score. The risk score may be, for example without limitation, a rating between one and ten or a score out of a hundred. In an example embodiment, various factors may be apportioned points in accordance with a scoring template. Table 1 below provides an exemplary scoring template:

TABLE 1

| Factor | Score |
|---|---|
| Current | N1 × 5 pts |
| prescribed | |
| medication | |
| count “N1” | |
| Current | N2 × 5 pts |
| per-condition | |
| prescribed | |
| medication | |
| count “N2” | |
| Current | N3 × 5 pts |
| co-morbidity | |
| count “N3” | |
| Current | Heart disease +10 pts |
| serious | Cancer +5 pts |
| condition | Chronic lower respiratory diseases +35 pts |
| profile | Stroke, cerebrovascular diseases +25 pts |
| | Alzheimer’s disease +50 pts |
| | Diabetes Type 1 +50 pts |
| | Diabetes Type 2 +5 pts |
| | Influenza & Pneumonia +50 pts |
| | Nephritis, Nephrotic Syndrome, and Nephrosis +30 pts |
| | Intentional Self-harm, Suicide +50 pts |
| Current | Medication for leading cause of death (LCOD) |
| medication | health condition taken twice or more daily +25 pts |
| titration | |
| profile | |
| Current | One or more incidents of emergency medical |
| medication | treatment in the past 10 years +50 pts |
| adherence | |
| emergency | |
| profile | |
| Current | Presence of one or more high risk genomes |
| high-risk | for a particular disease +10 pts |
| genomic | |
| profile | |

As noted above, the optimized delivery prediction system 101 utilizes the current medical history profile 402 as an input for the initial optimized drug need prediction iteration 408 and each post-initial optimized drug need prediction iteration 410A, 410B . . . N. In some embodiments, a current medical history profile 402 associated with a high risk score above a threshold (e.g., above 60 points) may lead to the target user profile being deemed ineligible for the optimized delivery prediction system 101. If the risk score associated with the current medical history profile 402 is below an predetermined threshold, then the optimized delivery prediction system 101 proceeds to perform the initial optimized drug need prediction iteration 408 as depicted in FIG. 4.

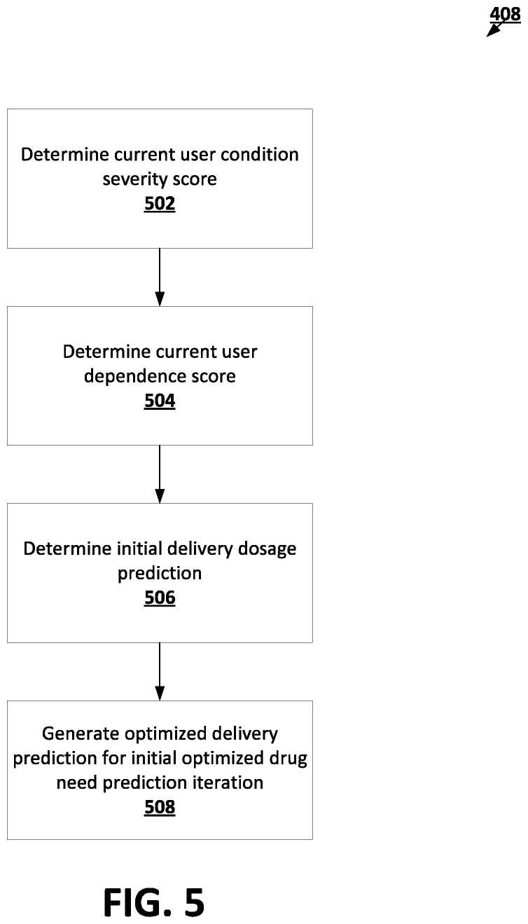

FIG. 5 provides a flowchart diagram illustrating an example process for performing an initial optimized drug need prediction iteration 408 by the optimized delivery prediction system 101.

At step/operation 502, the optimized delivery prediction system 101 determines a current user condition severity score for the target user profile. The current user condition severity score may describe a condition of a target user in relation to one or more current serious health conditions. In other words, the current user condition severity score can be an inferred determination as to whether the target user profile currently has one or more serious health conditions. The current user condition severity score may be determined based at least in part on the current medical history profile (e.g., the current serious condition profile) of the target user.

At step/operation 504, the optimized delivery prediction system 101 determines a current user dependence score for the target user profile with respect to the target drug profile. The current user dependence score can be an inferred determination about a target user's dependence on a drug at a delivery time. The current user dependence score may also be determined based at least in part on the current medical history profile of the target user. In some embodiments, the current user dependence score may be based at least in part on a current user condition severity score and a drug necessity profile. The current user condition severity score may describe/indicate one or more serious health conditions of a target user at a delivery time.

At step/operation 506, the optimized delivery prediction system 101 determines an initial delivery dosage prediction for the target user profile with respect to the target drug profile. The initial delivery dosage prediction describes one or more delivery parameters such as a recommended dosage of a drug provided on an initial delivery. For example, deliver five 10 mg dosages of a particular medication on September 1.

Then, at step/operation 508, the optimized delivery prediction system 101 generates an optimized delivery prediction for the initial optimized drug need prediction iteration. For example, the optimized delivery prediction system 101 generates one or more additional delivery parameters such as recommended timings and recommended dosages based at least in part on/subsequent to the initial delivery of the drug. In accordance with the preceding example, the target user is taking one 10 mg dosage of the medication daily, and the drug necessity score for the medication indicates that the drug is not necessary. Therefore, a second delivery of the medication can be provided on September 5 and a third delivery can be provided on September 10.

Returning to FIG. 4, the optimized delivery prediction system 101 is further configured to perform one or more post-initial optimized drug need prediction iterations 410A, 410B . . . N and a corresponding optimized delivery prediction 414A, 414B . . . N for each post-initial optimized drug need prediction iteration. 410A, 410B . . . N. As shown, the optimized delivery prediction system 101 may generate each post-initial optimized drug need prediction iteration 410A, 410B . . . N based at least in part on the initial optimized drug need prediction iteration 408 and/or previous optimized drug need prediction iterations, current medical history profile 402 for the target user profile, drug necessity profile 404, and current usage monitoring data 406 for the target user profile with respect to the target drug profile. The current usage monitoring data 406 may comprise drug usage data describing how often the target user has taken the medication after an initial delivery.

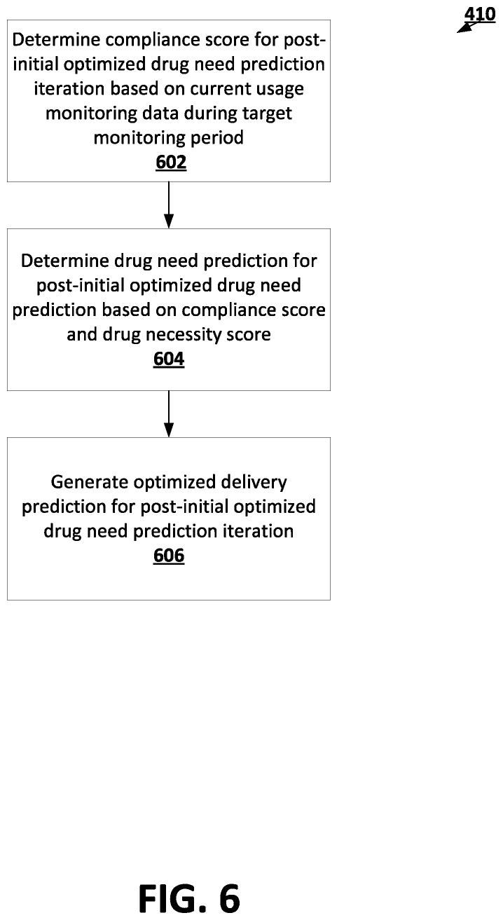

FIG. 6 provides a flowchart diagram illustrating an example process for performing a post-initial optimized drug need prediction iteration 410A, 410B . . . N by the optimized delivery prediction system 101. The post-initial delivery dosage prediction may be a recommended dosage of a drug provided on a subsequent delivery based at least in part on a drug necessity profile, current usage monitoring data (e.g., target user drug usage data) during a time period after the initial delivery, as well as a recommended dosage and recommended timing of a post-initial optimized delivery prediction.

As noted above, the post-initial delivery dosage prediction may be based at least in part on the current usage monitoring data which may comprise information/data describing whether a target user is taking a drug as prescribed (i.e., in accordance with a corresponding medical intake protocol/regimen).

As noted above, the post-initial delivery dosage prediction may include a drug need prediction describing an inferred conclusion as to whether or not a target user needs a drug at a particular prediction time (e.g., a time associated with performing an optimized drug need prediction iteration). The drug need prediction may be a negative drug need prediction or an affirmative drug need prediction. A negative drug need prediction may be an inferred conclusion that a target user does not currently need and/or will no longer need a drug. For example, the negative drug need prediction may correspond with a determination that a drug necessity score describes a sufficient lack of necessity condition (i.e., the target user does not currently and/or will no longer need a drug). A positive drug need prediction may be an inferred conclusion that a target user currently needs a drug and/or will need a drug.

Starting at step/operation 602, the optimized delivery prediction system 101 determines a compliance score for the post-initial optimized drug need prediction iteration based at least in part on current usage monitoring data during a target monitoring period. The compliance score may indicate whether or not, and to what degree, the target user has taken the target drug as prescribed during a time interval between two deliveries (e.g., the target user has taken only 50% of the total recommended dosage during the time interval thus far).

The current usage monitoring data may further describe one or more non-compliance events corresponding with the target user not taking a prescribed medication in accordance with a recommended medication intake protocol/regimen (e.g., taking a medication later than prescribed, missing a recommended dosage and/or the like). The compliance score may describe a sufficient non-compliance condition that satisfies one or more parameters, where satisfaction of the condition describes that the target user has not adhered to a recommended medication intake protocol/regimen. The sufficient non-compliance condition may correspond with length of time, a number of missed dosages and/or the like (e.g., the target user has met or exceeded a threshold number of non-compliance events with respect to the target drug). Different drugs may be associated with different parameters with respect to the sufficient non-compliance condition. For example, a sufficient non-compliance condition for a drug to treat a serious health condition may be a single dosage of the drug, whereas a sufficient non-compliance condition for a drug to treat a less serious health condition may be several dosages and/or a longer time interval.

Next, at step/operation 604, the optimized delivery prediction system 101 determines a drug need prediction for the post-initial optimized drug need prediction based at least in part on the compliance score and the drug necessity score. In other words, the optimized delivery prediction system 101 determines an inferred conclusion as to whether or not the user currently needs the drug. For example, if the compliance score satisfies a sufficient non-compliance condition and the drug necessity score indicates that the drug is not considered necessary for the target user, the optimized delivery prediction system 101 may determine that the target user no longer needs the drug.

At step/operation 606, the optimized delivery prediction system 101 generates the optimized delivery prediction for the post-initial optimized drug need prediction iteration. The optimized delivery prediction may comprise one or more modifications to the optimized delivery prediction for the initial optimized drug need prediction iteration. For example, the optimized delivery prediction for the post-initial optimized drug need prediction iteration may extend or truncate the interval until the next recommended delivery based at least in part on the determined drug need prediction. In some embodiments the optimized delivery prediction may discontinue further deliveries if the optimized delivery prediction system 101 determines that the target user no longer needs the drug.

Returning to FIG. 4, the optimized delivery prediction system 101 is configured to perform one or more post-initial optimized drug need prediction iterations 410A, 410B . . . N in order to generate corresponding optimized delivery predictions 414A, 414B . . . N. Each optimized delivery prediction 414A, 414B . . . N may include a post-initial delivery dosage prediction and post-initial delivery timing prediction.

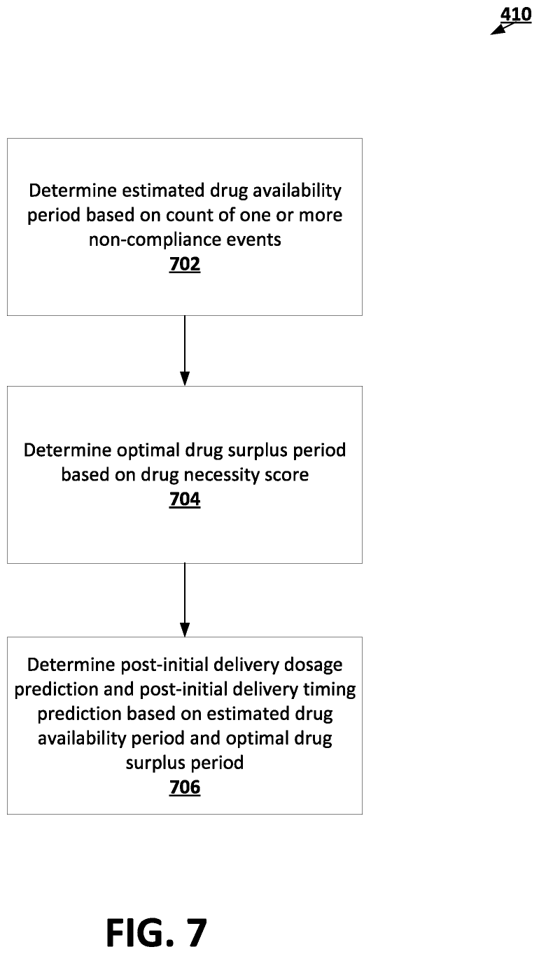

FIG. 7 provides a flowchart diagram illustrating an example process for determining a post-initial delivery dosage prediction and post-initial delivery timing prediction for a post-initial optimized drug prediction iteration.

At step/operation 702, the optimized delivery prediction system 101 determines an estimated drug availability period based at least in part on a count of one or more non-compliance events. The estimated drug availability period may be a time period for which the target user has a sufficient amount of medication left. The estimated drug availability period may be based at least in part on a count of one or more non-compliance events corresponding with whether or not the target user took a prescribed medication in accordance with a recommended medication intake protocol/regimen or missed a recommended dosage. For example, if a target user receives 5 dosages of a drug to be taken once a day the estimated drug availability period is 5 days. If the target user misses one dosage, the estimated drug availability period for the drug may be extended from 5 days to 6 days to correspond with the missed dosage.

Next, at step/operation 704, the optimized delivery prediction system 101 determines an optimal drug surplus period based at least in part on the current drug necessity score. The optimal drug surplus period may describe a recommended surplus period for which additional medication is provided. For instance, the optimal drug surplus period may indicate that a target user should always have an additional day's supply of the drug. The optimal drug surplus period may be based at least in part on the drug necessity score which indicates whether a drug is considered necessary based at least in part on one or more drug characteristics (e.g., severe side-effects, high-risk of potential abuse and/or the like) or user characteristics (e.g., user dependence factors).

Then, at step/operation 706, the optimized delivery prediction system 101 determines a post-initial delivery dosage prediction and post-initial delivery timing prediction based at least in part on the estimated drug availability period and the optimal drug surplus period. The estimated drug availability period may be a prospective time period corresponding with an assumed number of remaining dosages available to a target user out of a total number of dosages from an initial or subsequent delivery.

In some embodiments, the optimized delivery prediction system 101 may determine the post-initial delivery dosage prediction and post-initial delivery timing prediction based at least in part on a user availability profile. The term user availability profile may comprise information/data relating to the prospective location of a target user across a time period. The prospective location information/data may include data from the target user profile's schedule (e.g., calendar data describing prospective travel plans). For example, the prospective location information/data may indicate that a target user intends to travel during a prospective time period. Accordingly, it may be inferred that the target user will be unavailable to receive (e.g., access, collect and/or the like) a delivery during the prospective time period. A post-initial delivery dosage prediction may be based at least in part on the target user profile availability profile such that additional dosages of a drug may be provided in a delivery to account for the prospective time period when the target user is expected to be unavailable to receive a subsequent delivery of the drug.

Returning to FIG. 4, as further illustrated, the optimized delivery prediction system 101 is configured to continue to perform a plurality of subsequent post-initial optimized drug need prediction iterations 410A, 410B . . . N and generate corresponding post-initial optimized delivery predictions 414A, 414B . . . N based at least in part on the preceding optimized drug prediction iteration(s), current medical history profile 402, drug necessity profile 404, and current usage monitoring data 406. The optimized delivery prediction system 101 may be configured to periodically perform each subsequent post-initial optimized drug need prediction iteration 410A, 410B . . . N or in response to one or more triggers (e.g., based at least in part on information/data received from one or more client computing entities 102). In an example embodiment, the optimized prediction system 101 may be configured to perform a subsequent post-initial optimized drug need prediction iteration 410A, 410B . . . N in response to receiving new current usage monitoring data 406 that satisfies one or more parameters.

It will be appreciated that the invention is not limited to the number of iterations illustrated in FIG. 4. The optimized delivery prediction system 101 may be configured to continue to perform post-initial optimized drug prediction iterations periodically and/or based at least in part on predetermined triggers (e.g., updated current usage monitoring data 406 that satisfies various parameters).

### Generating User Interface Data

As noted above, the optimized delivery prediction system 101 may generate one or more outputs (e.g., optimized delivery predictions) and provide (e.g., send, transmit, and/or the like) the outputs to one or more client computing entities 102 for generating user interface data and/or dynamically updating a user interface.

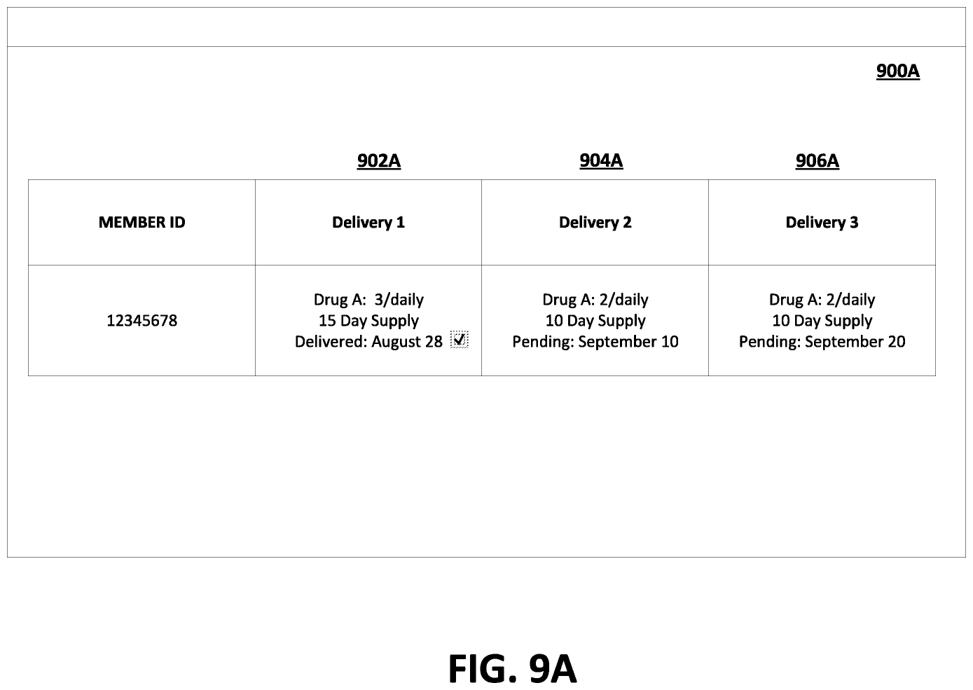

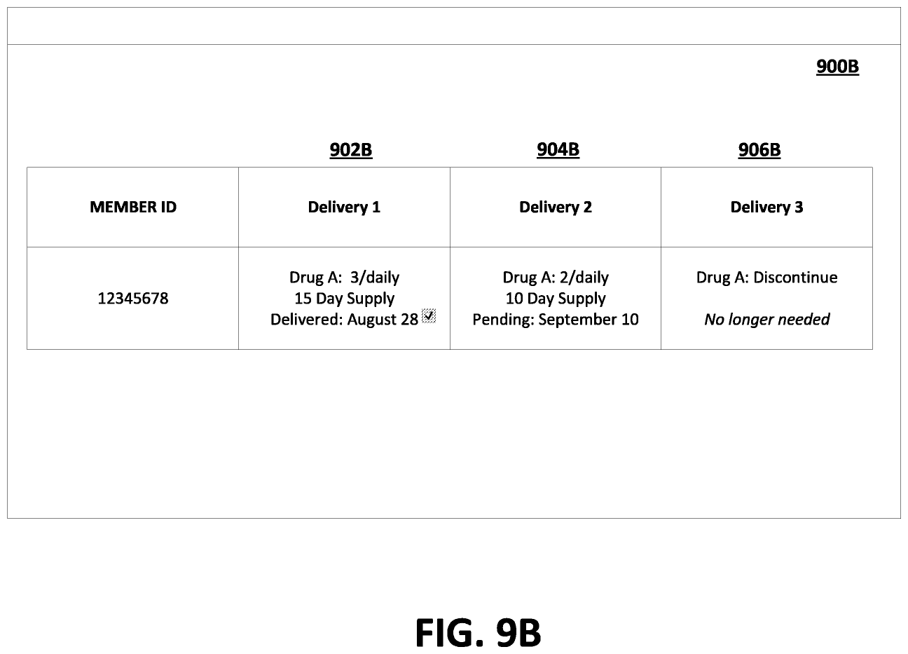

FIGS. 9A-B provide an operational example of dynamically updating user interface data based at least on an initial optimized drug need prediction iteration and a post-initial optimized drug need prediction iteration, respectively. In various embodiments, the client computing entity 102 generates user interface data (e.g., one or more data objects) which is provided (e.g., transmitted, sent and/or the like) for presentation by the user interface 900A-B of a user computing entity and/or client computing entity 102. The user interface 900A-B may comprise various features and functionality for accessing, and/or viewing data objects and/or alerts. The user interface 900A-B may also comprise messages in the form of banners, headers, notifications, and/or the like.

As illustrated in FIG. 9A, an example user interface 900A may receive user interface data for presentation based at least in part on at least an initial optimized drug need prediction iteration. As shown, the user interface data comprises an indication of an initial historical drug delivery 902A and subsequent recommended (i.e., prospective) drug deliveries 904A, 906A, with respect to a target user, based at least in part on the initial optimized drug need prediction iteration. Each of the historical drug delivery 902A and recommended drug deliveries 904A, 906A comprises prescription information for a drug, a recommended dosage/period, and a historical or recommended delivery date.

Referring to FIG. 9B, based at least in part on a subsequent post-initial optimized drug need prediction iteration, the target user profile interface 900B may receive additional/modified user interface data for presentation (e.g., for dynamically updating the user interface 900B of the user computing entity and/or client computing entity). As shown, the user interface data comprises an indication of the initial historical drug delivery 902B (identical to 902A), and modified subsequent recommended deliveries 904B, 906B with respect to the target user, based at least in part on the post-initial optimized drug need prediction iteration comprising new information/data. Each of the historical drug delivery 902B and recommended drug deliveries 904B, 906B comprises prescription information for the drug, a recommended dosage/period, and a historical or recommended delivery date. As shown, the information (recommended dosage and recommended delivery) associated with the second delivery 904A in FIG. 9A is identical to the information associated with the second delivery 904B in FIG. 9B. However, the information associated with the third delivery 906A in FIG. 9A is different from the information associated with the third delivery in FIG. 9B and indicates that the third delivery should be canceled as the drug is no longer needed.

### Exemplary User Registration Process

In some embodiments, a target user may register to utilize and/or participate in various aspects of the optimized delivery prediction system 101. For instance, the target user may register for a delivery program provided by a registered pharmacy by opting to receive portions of a prescribed medication periodically. Upon termination of deliveries, unused medication can be optimally apportioned by the optimized delivery prediction system 101 and/or pharmacy (e.g., based at least in part on user preferences/selections).

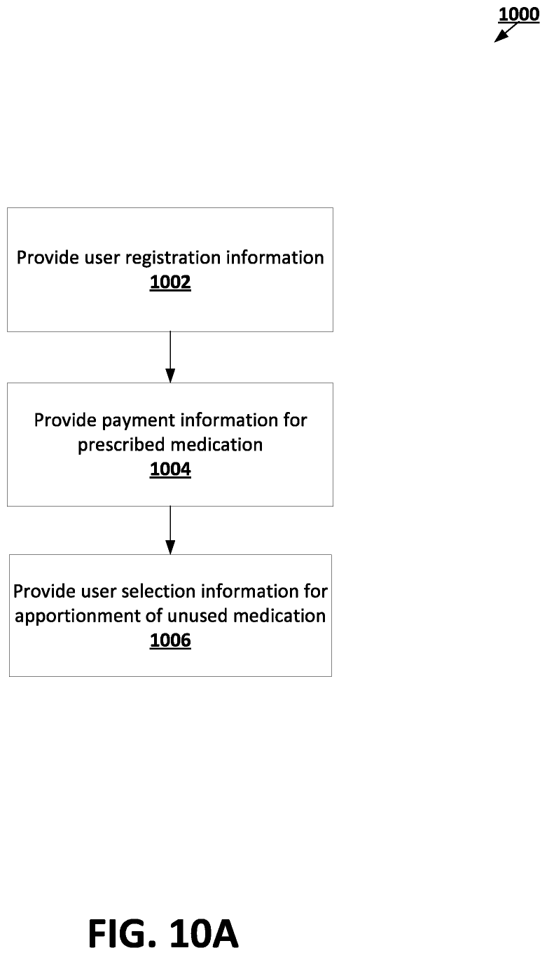

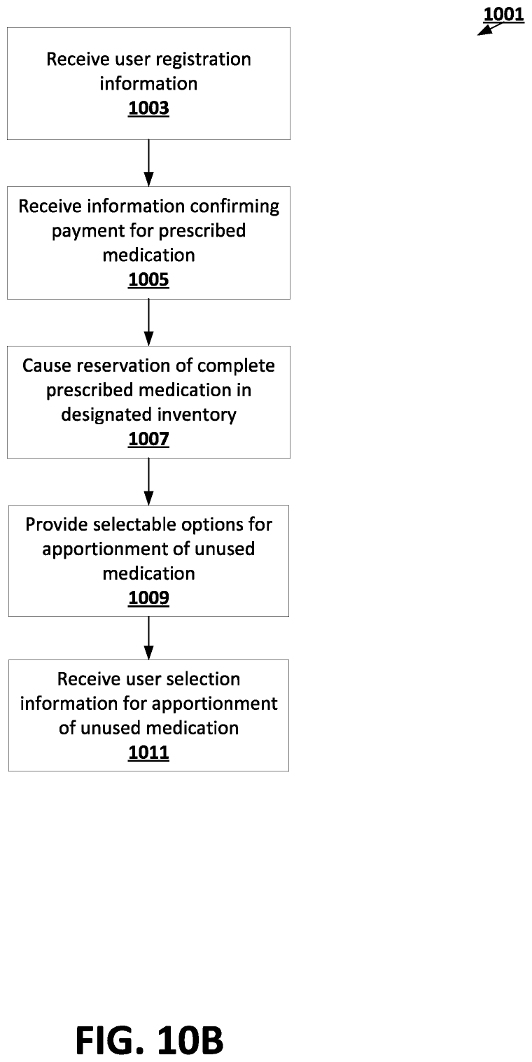

FIG. 10A provides a flowchart diagram illustrating an example user registration process 1000 by a user computing entity (e.g., via the client computing entity 102) and FIG. 10B provides a flowchart diagram illustrating an example user registration process 1001 by the optimized delivery prediction system 101, in accordance with some embodiments discussed herein.

Referring to FIG. 10A, at step/operation 1002, a target user may provide (e.g., send, transmit and/or the like) user registration information (e.g., via the target user profile interface of a user computing entity/client computing entity 102). In some embodiments, the registration information may be intended for a particular pharmacy, cooperative and/or the like.

Then, referring again to FIG. 10B, at step/operation 1003, the optimized delivery prediction system 101 receives the target user profile registration information provided by the target user.

Returning to FIG. 10A, at step/operation 1004, the target user, via the target user profile computing entity, provides and/or confirms payment information for a prescribed medication. For example, the target user may submit a co-payment amount.

Returning to FIG. 10B, at step/operation 1005, the optimized delivery prediction system 101 receives the payment information confirming payment for the prescribed medication.

Then, at step/operation 1007, the optimized delivery prediction system 101 causes reservation of a complete prescribed medication in designated inventory. The designated inventory may be located at the pharmacy/cooperative, physically separate or provided by a third party.

At step/operation 1009, the optimized delivery prediction system 101 provides selectable options for apportionment of unused medication. The selectable options may include, allowing the pharmacy to redistribute unused portions of medication, receiving a rebate or credit for an unused portion of medication or a pay-as-you-go option which may not cause/result in reservation of the complete prescribed medication. In some embodiments, the pay-as-you go option may only be available for target drugs that satisfy certain parameters (e.g., corresponding with a drug necessity score falling within a particular range). In some embodiments, a less available medication may attract a higher rebate or credit incentive.

Returning to FIG. 10A, at step/operation 1006, the target user of the target user profile computing entity provides user selection information for apportionment of unused medication upon termination of deliveries.

Then, referring again to FIG. 10B, at step/operation 1011, the optimized delivery prediction system 101 receives the target user profile selection information for apportionment of unused medication. In some embodiments, the optimized delivery prediction system 101 determines an optimal apportionment of unused medication. The optimal apportionment of unused medication may be based at least in part on, for example, without limitation, one or more of third-party organizational needs, target user historical data (e.g., previous selections/distributions), logistical constraints, target user preferences and/or the like.

### Exemplary Optimized Drug Delivery

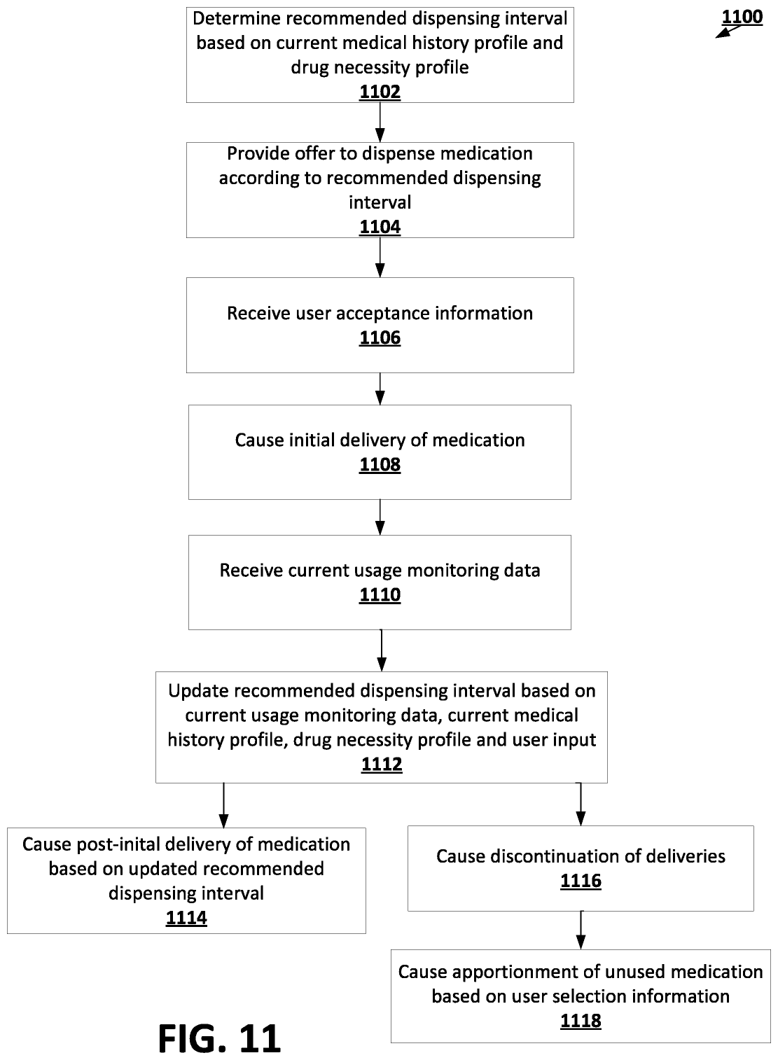

FIG. 11 provides a flowchart diagram illustrating an example process 1100 for performing an optimized drug delivery by an optimized delivery prediction system 101 in accordance with some embodiments discussed herein.

At step/operation 1102, the optimized delivery prediction system 101 determines a recommended dispensing interval based at least in part on the current medical history profile and drug necessity profile. In various embodiments, the current medical history profile may include target user profile motility, symptom severity, geographic location, length of prescription and/or the like. Additionally and/or alternatively, a prescribing clinician (e.g., medical doctor or pharmacist) may provide additional data/information to the optimized delivery prediction system 101. For example, the prescribing clinician may be aware of contraindications and provide additional data/information pertaining to a safe recommended dispensing interval. The optimized delivery prediction system 101 may also utilize other available information about relevant standardized dispensing/default intervals (e.g., based at least in part on demographic information/data).

Next, at step/operation 1104, the optimized delivery prediction system 101 provides an offer to dispense medication according to the recommended dispensing interval. The offer may be provided for display by a user computing entity/client computing entity 102.

At step/operation 1106, the optimized delivery prediction system 101 receives user acceptance information in relation to the offer to dispense medication according to the recommended dispensing interval. The acceptance information may be provided by the target user profile computing entity/client computing entity 102. In some embodiments, the target user may modify or request modifications to the recommended dispensing interval. The optimized delivery prediction system 101 may further verify that such modifications/requests are within range of accepted parameters for the target user. The target user may also agree to provide usage monitoring data with respect to the medication by manually providing information via an application, digital assistant, text messages and/or the like. The usage monitoring data may also be provided automatically via Internet of Things (IoT) medication devices and methods, or using various health-tracking applications.

At step/operation 1108, the optimized delivery prediction system 101 causes an initial delivery of medication. The optimized delivery prediction system 101 may log the available balance to a reserved inventory and/or schedule a subsequent delivery.

At step/operation 1110, the optimized delivery prediction system 101 receives current usage monitoring data for the target user.

At step/operation 1112, the optimized delivery prediction system 101 updates the recommended dispensing interval based at least in part on the current usage monitoring data, current medical history profile, the drug necessity profile and/or user input. The optimized delivery prediction system 101 may update the recommended dispensing interval and/or modify one or more scheduled deliveries based at least in part on the current usage monitoring data. For example, if the current usage monitoring data indicates that the dosage intake is normal, the optimized delivery prediction system 101 may confirm the next delivery.

At step/operation 1114, the optimized delivery prediction system 101 causes a post-initial delivery of medication based at least in part on the updated recommended dispensing interval. For example, if the dosage intake is determined to be reduced in the preceding step, the optimized delivery prediction system 101 may delay the next delivery and generate, e.g., via the client computing entity 102, a system notification for the target user.

Alternatively, at step/operation 1116, the optimized delivery prediction system 101 causes discontinuation of deliveries. For example, if the dosage intake has stopped and the optimized delivery prediction system 101 determines that the medication is no longer needed, and/or the target user confirms that the medication is no longer needed, the optimized delivery prediction system 101 may cause cancelation of further scheduled deliveries.

At step/operation 1118, the optimized delivery prediction system 101 causes apportionment of unused medication based at least in part on the received user selection information and/or a determined optimal apportionment.

### VI. CONCLUSION

Many modifications and other embodiments will come to mind to one skilled in the art to which this disclosure pertains having the benefit of the teachings presented in the foregoing descriptions and the associated drawings. Therefore, it is to be understood that the disclosure is not to be limited to the specific embodiments disclosed and that modifications and other embodiments are intended to be included within the scope of the appended claims. Although specific terms are employed herein, they are used in a generic and descriptive sense only and not for purposes of limitation.

1. A computer-implemented method for performing optimized drug delivery for a target drug profile with respect to a target user profile, the computer-implemented method comprising:
performing, by one or more processors, a plurality of optimized drug need prediction iterations, wherein:
each optimized drug need prediction iteration of the plurality of optimized drug need predictions is configured to generate an optimized delivery prediction for the optimized drug need prediction iteration,
generating the optimized delivery prediction for an initial optimized drug need prediction iteration of the plurality of optimized drug need predictions comprises: (i) determining an initial delivery dosage prediction for the initial optimized drug need prediction iteration based at least in part on a current medical history profile of the target user profile at a prediction time of the initial optimized drug need prediction iteration and of the target drug profile in relation to the target user profile, and (ii) generating the optimized delivery prediction for the initial optimized drug need prediction iteration based at least in part on the initial delivery dosage prediction, and
generating each optimized delivery prediction for a post-initial optimized drug need prediction iteration of the plurality of optimized drug need predictions comprises: (i) determining a drug need prediction for the prediction time of the post-initial optimized drug need prediction iteration based at least in part on the drug necessity profile and current usage monitoring data for the target user profile in relation to the target drug profile during a target monitoring period for the post-initial optimized drug need prediction, (ii) in response to determining that the drug need prediction describes an affirmative drug need prediction, generating the optimized delivery prediction based at least in part on a post-initial delivery dosage prediction for the post-initial optimized drug need prediction iteration and a post-initial delivery timing prediction for the post-initial optimized drug need prediction iteration, and (iii) in response to determining that the drug need prediction describes a negative drug need prediction, generating the optimized delivery prediction based at least in part on the negative drug need prediction;
performing, by the one or more processors, the optimized drug delivery based at least in part on each optimized delivery prediction for an optimized drug need prediction iteration of the plurality of optimized drug need predictions.

2. The computer-implemented method of claim 1, wherein performing the initial optimized drug need prediction iteration of the plurality of optimized drug need prediction iterations comprises:
determining a current user condition severity score for the initial optimized drug need prediction iteration based at least in part on the current medical history profile of the target user profile at the prediction time of the initial optimized drug need prediction iteration;
determining a current user dependence score for the initial optimized drug need prediction iteration based at least in part on the current user condition severity score and the drug necessity profile;
determining the initial delivery dosage prediction for the initial optimized drug need prediction iteration based at least in part on the current user dependence score; and
generating the optimized delivery prediction for the initial optimized drug need prediction iteration based at least in part on the initial delivery dosage prediction.

3. The computer-implemented method of claim 1, wherein performing each post-initial optimized drug need prediction iteration of the plurality of optimized drug need prediction iterations comprises:
determining a compliance score for the post-initial optimized drug need prediction iteration based at least in part on the current usage monitoring data for the target user profile in relation to the target drug profile during the target monitoring period for the post-initial optimized drug need prediction; and
determining the drug need prediction for the post-initial optimized drug need prediction iteration based at least in part on the compliance score and the drug necessity score.

4. The computer-implemented method of claim 3, wherein determining the drug need prediction for the post-initial optimized drug need prediction iteration further comprises:
in response to determining that the compliance score describes a sufficient non-compliance condition and the drug necessity score describes a sufficient lack of necessity condition, determining the negative drug need prediction.

5. The computer-implemented method of claim 3, wherein performing the post-initial optimized drug need prediction iteration of the plurality of optimized drug need prediction iterations comprises:
in response to determining that the drug need prediction describes the positive drug need prediction, the compliance score describes one or more non-compliance events, and the drug necessity score describes a sufficient lack of necessity condition, determining the post-initial delivery dosage prediction for the post-initial optimized drug need prediction iteration based at least in part on a count of the one or more non-compliance events.

6. The computer-implemented method of claim 3, wherein performing the post-initial optimized drug need prediction iteration of the plurality of optimized drug need prediction iterations comprises:
in response to determining that the drug need prediction describes the positive drug need prediction and the compliance score describes one or more non-compliance events:
determining an estimated drug availability period based at least in part on a count of the one or more non-compliance events,
determining an optimal drug surplus period based at least in part on the drug necessity score, and
determining the post-initial delivery dosage prediction for the post-initial optimized drug need prediction iteration based at least in part on the estimated drug availability period and the optimal drug surplus period.

7. The computer-implemented method of claim 6, wherein determining the post-initial delivery dosage prediction for the post-initial optimized drug need prediction iteration is further performed based at least in part on a user availability profile of the target user profile.

8. The computer-implemented method of claim 1, wherein the current medical history profile of the target user profile comprises:
a current prescribed medication count of the target user profile,
a current per-condition prescribed medication count of the target user profile,
a current co-morbidity count of the target user profile,
a current serious condition profile of the target user profile,
a current medication titration profile of the target user profile,
a current medication adherence emergency profile of the target user profile, and
a current high-risk genomic profile of the target user profile.

9. An apparatus for performing optimized drug delivery for a target profile with respect to a target user profile, the apparatus comprising at least one processor and at least one memory including program code, the at least one memory and the program code configured to, with the processor, cause the apparatus to at least:
perform a plurality of optimized drug need prediction iterations, wherein:
each optimized drug need prediction iteration of the plurality of optimized drug need predictions is configured to generate an optimized delivery prediction for the optimized drug need prediction iteration,
generating the optimized delivery prediction for an initial optimized drug need prediction iteration of the plurality of optimized drug need predictions comprises: (i) determining an initial delivery dosage prediction for the initial optimized drug need prediction iteration based at least in part on a current medical history profile of the target user profile at a prediction time of the initial optimized drug need prediction iteration and of the target drug profile in relation to the target user profile, and (ii) generating the optimized delivery prediction for the initial optimized drug need prediction iteration based at least in part on the initial delivery dosage prediction, and
generating each optimized delivery prediction for a post-initial optimized drug need prediction iteration of the plurality of optimized drug need predictions comprises: (i) determining a drug need prediction for the prediction time of the post-initial optimized drug need prediction iteration based at least in part on the drug necessity profile and current usage monitoring data for the target user profile in relation to the target drug profile during a target monitoring period for the post-initial optimized drug need prediction, (ii) in response to determining that the drug need prediction describes an affirmative drug need prediction, generating the optimized delivery prediction based at least in part on a post-initial delivery dosage prediction for the post-initial optimized drug need prediction iteration and a post-initial delivery timing prediction for the post-initial optimized drug need prediction iteration, and (iii) in response to determining that the drug need prediction describes a negative drug need prediction, generating the optimized delivery prediction based at least in part on the negative drug need prediction;
perform the optimized drug delivery based at least in part on each optimized delivery prediction for an optimized drug need prediction iteration of the plurality of optimized drug need predictions.

10. The apparatus of claim 9, wherein the program code is further configured to, with the processor, cause the apparatus at least to:
perform the initial optimized drug need prediction iteration of the plurality of optimized drug need prediction iterations by:
determining a current user condition severity score for the initial optimized drug need prediction iteration based at least in part on the current medical history profile of the target user profile at the prediction time of the initial optimized drug need prediction iteration;
determining a current user dependence score for the initial optimized drug need prediction iteration based at least in part on the current user condition severity score and the drug necessity profile;
determining the initial delivery dosage prediction for the initial optimized drug need prediction iteration based at least in part on the current user dependence score; and
generating the optimized delivery prediction for the initial optimized drug need prediction iteration based at least in part on the initial delivery dosage prediction.

11. The apparatus of claim 9, wherein the program code is further configured to, with the processor, cause the apparatus at least to:
perform each post-initial optimized drug need prediction iteration of the plurality of optimized drug need prediction iterations by:
determining a compliance score for the post-initial optimized drug need prediction iteration based at least in part on the current usage monitoring data for the target user profile in relation to the target drug profile during the target monitoring period for the post-initial optimized drug need prediction; and
determining the drug need prediction for the post-initial optimized drug need prediction iteration based at least in part on the compliance score and the drug necessity score.

12. The apparatus of claim 11, wherein the program code is further configured to, with the processor, cause the apparatus at least to:
determine the drug need prediction for the post-initial optimized drug need prediction iteration by:
in response to determining that the compliance score describes a sufficient non-compliance condition and the drug necessity score describes a sufficient lack of necessity condition, determining the negative drug need prediction.

13. The apparatus of claim 11, wherein the program code is further configured to, with the processor, cause the apparatus at least to:
perform the post-initial optimized drug need prediction iteration of the plurality of optimized drug need prediction iterations by:
in response to determining that the drug need prediction describes the positive drug need prediction, the compliance score describes one or more non-compliance events, and the drug necessity score describes a sufficient lack of necessity condition, determining the post-initial delivery dosage prediction for the post-initial optimized drug need prediction iteration based at least in part on a count of the one or more non-compliance events.

14. The apparatus of claim 11, wherein the program code is further configured to, with the processor, cause the apparatus at least to:
perform the post-initial optimized drug need prediction iteration of the plurality of optimized drug need prediction iterations by:
in response to determining that the drug need prediction describes the positive drug need prediction and the compliance score describes one or more non-compliance events:
determining an estimated drug availability period based at least in part on a count of the one or more non-compliance events,
determining an optimal drug surplus period based at least in part on the drug necessity score, and
determining the post-initial delivery dosage prediction for the post-initial optimized drug need prediction iteration based at least in part on the estimated drug availability period and the optimal drug surplus period.

15. A computer program product for performing optimized drug delivery for a target drug profile with respect to a target user profile, the computer program product comprising at least one non-transitory computer-readable storage medium having computer-readable program code portions stored therein, the computer-readable program code portions configured to:
perform a plurality of optimized drug need prediction iterations, wherein:
each optimized drug need prediction iteration of the plurality of optimized drug need predictions is configured to generate an optimized delivery prediction for the optimized drug need prediction iteration,
generating the optimized delivery prediction for an initial optimized drug need prediction iteration of the plurality of optimized drug need predictions comprises: (i) determining an initial delivery dosage prediction for the initial optimized drug need prediction iteration based at least in part on a current medical history profile of the target user profile at a prediction time of the initial optimized drug need prediction iteration and of the target drug profile in relation to the target user profile, and (ii) generating the optimized delivery prediction for the initial optimized drug need prediction iteration based at least in part on the initial delivery dosage prediction, and
generating each optimized delivery prediction for a post-initial optimized drug need prediction iteration of the plurality of optimized drug need predictions comprises: (i) determining a drug need prediction for the prediction time of the post-initial optimized drug need prediction iteration based at least in part on the drug necessity profile and current usage monitoring data for the target user profile in relation to the target drug profile during a target monitoring period for the post-initial optimized drug need prediction, (ii) in response to determining that the drug need prediction describes an affirmative drug need prediction, generating the optimized delivery prediction based at least in part on a post-initial delivery dosage prediction for the post-initial optimized drug need prediction iteration and a post-initial delivery timing prediction for the post-initial optimized drug need prediction iteration, and (iii) in response to determining that the drug need prediction describes a negative drug need prediction, generating the optimized delivery prediction based at least in part on the negative drug need prediction;
perform the optimized drug delivery based at least in part on each optimized delivery prediction for an optimized drug need prediction iteration of the plurality of optimized drug need predictions.

16. The computer program product of claim 15, wherein the computer-readable program code portions are further configured to:
perform the initial optimized drug need prediction iteration of the plurality of optimized drug need prediction iterations by:
determining a current user condition severity score for the initial optimized drug need prediction iteration based at least in part on the current medical history profile of the target user profile at the prediction time of the initial optimized drug need prediction iteration;
determining a current user dependence score for the initial optimized drug need prediction iteration based at least in part on the current user condition severity score and the drug necessity profile;
determining the initial delivery dosage prediction for the initial optimized drug need prediction iteration based at least in part on the current user dependence score; and
generating the optimized delivery prediction for the initial optimized drug need prediction iteration based at least in part on the initial delivery dosage prediction.

17. The computer program product of claim 15, wherein the computer-readable program code portions are further configured to:
perform each post-initial optimized drug need prediction iteration of the plurality of optimized drug need prediction iterations by:
determining a compliance score for the post-initial optimized drug need prediction iteration based at least in part on the current usage monitoring data for the target user profile in relation to the target drug profile during the target monitoring period for the post-initial optimized drug need prediction; and
determining the drug need prediction for the post-initial optimized drug need prediction iteration based at least in part on the compliance score and the drug necessity score.

18. The computer program product of claim 17, wherein the computer-readable program code portions are further configured to:
determine the drug need prediction for the post-initial optimized drug need prediction iteration by:
in response to determining that the compliance score describes a sufficient non-compliance condition and the drug necessity score describes a sufficient lack of necessity condition, determining the negative drug need prediction.

19. The computer program product of claim 17, wherein the computer-readable program code portions are further configured to:
perform the post-initial optimized drug need prediction iteration of the plurality of optimized drug need prediction iterations by:
in response to determining that the drug need prediction describes the positive drug need prediction, the compliance score describes one or more non-compliance events, and the drug necessity score describes a sufficient lack of necessity condition, determining the post-initial delivery dosage prediction for the post-initial optimized drug need prediction iteration based at least in part on a count of the one or more non-compliance events.

20. The computer program product of claim 17, wherein the computer-readable program code portions are further configured to:
perform the post-initial optimized drug need prediction iteration of the plurality of optimized drug need prediction iterations by:
in response to determining that the drug need prediction describes the positive drug need prediction and the compliance score describes one or more non-compliance events:
determining an estimated drug availability period based at least in part on a count of the one or more non-compliance events,
determining an optimal drug surplus period based at least in part on the drug necessity score, and
determining the post-initial delivery dosage prediction for the post-initial optimized drug need prediction iteration based at least in part on the estimated drug availability period and the optimal drug surplus period.
